(19) United States
(12) Patent Application Publication
(10) Pub. No.: US 2026/0272307 A1
(43) Pub. Date: Sep. 17, 2026

(54) PHOTOACOUSTIC DEVICES AND SYSTEMS

(71) Applicant: QUALCOMM Incorporated, San Diego, CA (US)

(72) Inventors: Yipeng LU, Moraga, CA (US); Hrishikesh Vijaykumar PANCHAWAGH, Cupertino, CA (US); Nicholas BUCHAN, San Jose, CA (US); Kostadin Dimitrov DJORDJEV, Los Gatos, CA (US); Camilo PEREZ SAAIBI, Dublin, CA (US); Legardo REYES, Fremont, CA (US); Ali LOPEZ, Dublin, CA (US); Ila BADGE, San Jose, CA (US); Chin-Jen TSENG, Fremont, CA (US); Ye ZHAN, Buffalo, NY (US); John Keith SCHNEIDER, Williamsville, NY (US)

(21) Appl. No.: 19/670,993

(22) Filed: May 7, 2026

Related U.S. Application Data
(62) Division of application No. 18/069,859, filed on Dec. 21, 2022.

(51) Int. Cl. A61B 5/00; A61B 5/021; B06B 1/06; B06B 3/00; G01H 11/08

(52) U.S. Cl. CPC A61B 5/0095; A61B 5/02125; A61B 5/6824; A61B 5/6826; B06B 1/0611; B06B 3/00; G01H 11/08; B06B 2201/76

(57) ABSTRACT

An apparatus may include a platen, a light source system and an ultrasonic receiver system. The light source system may be configured to provide light to a target object on an outer surface of the platen. The light source system may be configured to direct the light along a first axis oriented at a first angle relative to the outer surface of the platen. The ultrasonic receiver system may be configured to receive ultrasonic waves generated by the target object responsive to the light from the light source system. The ultrasonic receiver system may include one or more receiver elements residing in a receiver plane. A normal to the receiver plane being may be oriented along a second axis at a second angle relative to the outer surface of the platen. The first angle may be different from the second angle.

### CROSS-REFERENCE TO RELATED APPLICATIONS

This application claims priority to U.S. patent application Ser. No. 18/069,859, filed Dec. 21, 2022, entitled “PHOTOACOUSTIC DEVICES AND SYSTEMS,” and is related to U.S. patent application Ser. No. 18/069,901 (Attorney Docket Nos. 2300761/QUALP591US), entitled “PHOTOACOUSTIC DEVICES AND SYSTEMS INCLUDING ONE OR MORE LIGHT GUIDE COMPONENTS,” to U.S. patent application Ser. No. 18/069,877 (Attorney Docket Nos. 2205722U1/QUALP580AUS), entitled “SEMI-COMPACT PHOTOACOUSTIC DEVICES AND SYSTEMS,” to U.S. patent application Ser. No. 18/069,882 (Attorney Docket Nos. 2205722U2/QUALP580BUS), entitled “SEMI-COMPACT PHOTOACOUSTIC DEVICES AND SYSTEMS,” to U.S. patent application Ser. No. 18/069,885 (Attorney Docket Nos. 2205722U3/QUALP580CUS), entitled “SEMI-COMPACT PHOTOACOUSTIC DEVICES AND SYSTEMS,” to U.S. patent application Ser. No. 18/069,888 (Attorney Docket Nos. 2205722U4/QUALP580DUS), entitled “SEMI-COMPACT PHOTOACOUSTIC DEVICES AND SYSTEMS” and to U.S. patent application Ser. No. 18/069,893 (Attorney Docket Nos. 2205722U5/QUALP580EUS), entitled “SEMI-COMPACT PHOTOACOUSTIC DEVICES AND SYSTEMS,” all of which are hereby incorporated by reference in their entireties and for all purposes.

### TECHNICAL FIELD

This disclosure relates generally to photoacoustic devices and systems.

### DESCRIPTION OF RELATED TECHNOLOGY

A variety of different sensing technologies and algorithms are being implemented in devices for various biometric and biomedical applications, including health and wellness monitoring. This push is partly a result of the limitations in the usability of traditional measuring devices for continuous, noninvasive and ambulatory monitoring. Some such devices are, or include, photoacoustic devices. Although some previously-deployed photoacoustic devices and systems can provide acceptable results, improved photoacoustic devices and systems would be desirable.

### SUMMARY

The systems, methods and devices of this disclosure each have several aspects, no single one of which is solely responsible for the desirable attributes disclosed herein.

One innovative aspect of the subject matter described in this disclosure can be implemented in an apparatus. The apparatus may include a platen, a light source system and a receiver system. The receiver system may be, or may include, an ultrasonic receiver system. In some implementations, a mobile device (such as a wearable device, a cellular telephone, etc.) may be, or may include, at least part of the apparatus.

The light source system may be configured to provide light to a target object on an outer surface of the platen. The light source system may be configured to direct the light along a first axis oriented at a first angle relative to the outer surface of the platen.

The ultrasonic receiver system may be configured to receive ultrasonic waves generated by the target object responsive to the light from the light source system. The ultrasonic receiver system may include one or more receiver elements residing in a receiver plane. A normal to the receiver plane may be oriented along a second axis at a second angle relative to the outer surface of the platen.

In some examples, the second angle may be approximately 90 degrees. In some alternative examples, the second angle may be in a range from 20 degrees to 50 degrees.

According to some examples, at least a portion of the platen may be configured to direct the ultrasonic waves generated by the target object along a third axis that is at a third angle relative to the outer surface of the platen. In some examples, the third axis may be parallel, or substantially parallel, to the second axis. In some examples, the third angle may be different from the first angle.

In some examples, the light source system may include one or more light-directing elements configured to direct light from the light source system towards the target object along the first axis. In some such examples, the one or more light-directing elements may include a diffraction grating. In some examples, the one or more light-directing elements may include a lens.

According to some examples, the light source system may include a light source system surface having a normal that is parallel, or substantially parallel, to the first axis. In some such examples, a light source of the light source system resides on the light source system surface.

In some examples, a first platen portion residing between the light source system and the outer surface of the platen may have a first platen portion thickness that is less than a second platen portion thickness of a second platen portion residing between at least one receiver element of the ultrasonic receiver system and the outer surface of the platen. In some alternative examples, a first platen portion residing between the light source system and the outer surface of the platen may have a first platen portion thickness that is greater than a second platen portion thickness of a second platen portion residing between at least one receiver element of the ultrasonic receiver system and the outer surface of the platen.

According to some examples, the apparatus may include an acoustic waveguide configured to direct ultrasonic waves toward at least one receiver element of the ultrasonic receiver system. In some examples, a first distance traversed through the platen by the light from the light source system may be less than a second distance traversed by the ultrasonic waves through the platen and to at least one receiver element of the ultrasonic receiver system. According to some examples, the platen may include a recessed area configured to receive a digit.

In some examples, a platen portion configured to receive the light from the light source system may also be configured to reflect the ultrasonic waves generated by the target object towards at least one receiver element of the ultrasonic receiver system. In some such examples, a first distance traversed by light from the light source system through the platen portion to the target object may be less than a second distance traversed by the ultrasonic waves from the platen portion to at least one receiver element of the ultrasonic receiver system.

According to some examples, the first axis may be perpendicular to, or substantially perpendicular to, the outer surface. at least a portion of the platen may be configured to direct the ultrasonic waves generated by the target object along a third axis that is at a third angle relative to the outer surface of the platen. In some examples, the second axis may be parallel, or substantially parallel, to the third axis. In some examples, the first angle may be different from the third angle. In some such examples, a first portion of the platen may be configured to direct a first portion of the ultrasonic waves generated by the target object along the third axis and a second portion of the platen may be configured to direct a second portion of the ultrasonic waves generated by the target object along a fourth axis at a fourth angle relative to the outer surface. According to some examples, the third angle may be different from the first angle and the fourth angle.

In some examples, the ultrasonic receiver system may include a first receiver element residing in a first receiver plane that is substantially perpendicular to the third axis. In some such examples, the ultrasonic receiver system may include and a second receiver element residing in a second receiver plane that is substantially perpendicular to the fourth axis.

According to some examples, at least a portion of the platen may be configured to direct the ultrasonic waves generated by the target object along a third axis that is at a third angle relative to the outer surface of the platen. In some such examples, the third axis may be perpendicular to, or substantially perpendicular to, the outer surface. In some examples, the second axis may be parallel, or substantially parallel, to the third axis. In some examples, the first angle may be different from the third angle. According to some examples, the light source system may include a first light source portion configured to direct first light towards the target object along the first axis and a second light source portion configured to direct second light towards the target object along a fourth axis at a fourth angle relative to the outer surface of the platen. In some such examples, the third angle may be different from the first angle and the fourth angle.

In some examples, the apparatus may include acoustic isolation material residing between the light source system and at least a portion of the ultrasonic receiver system. According to some examples, the apparatus may include electromagnetic noise suppression material proximate at least a portion of the ultrasonic receiver system, proximate conductive material attached to at least a portion of the ultrasonic receiver system, or a combination thereof.

According to some examples, the apparatus may be configured for attachment to a human wrist. In some such examples, the light source system may be configured to provide light to one or more arteries within the human wrist. In some examples, the light source system may be configured to provide light to one or more arteries within a human finger.

According to some examples, the platen, the light source system, or a combination thereof, may be configured for transmitting light in a range from 400 to 1000 nanometers. In some examples, the light source system may be configured to provide pulses of light having pulse widths in a range from 50 to 500 nanoseconds.

In some examples, at least a portion of the ultrasonic receiver system may include a composite piezoelectric material. According to some examples, at least a portion of the ultrasonic receiver system may include a conductive layer, a first piezoelectric layer proximate a first side of the conductive layer and a second piezoelectric layer proximate a second side of the conductive layer. In some such examples, the first piezoelectric layer and the second piezoelectric layer may include a piezoelectric copolymer, a piezoelectric composite, or a combination thereof. In some examples, the apparatus may include a first electrical grounding layer portion and a second electrical grounding layer portion. In some such examples, the first piezoelectric layer may reside between the first electrical grounding layer portion and the conductive layer and the second piezoelectric layer resides between the second electrical grounding layer portion and the conductive layer.

According to some examples, at least a portion of the platen may have an acoustic impedance that is configured to approximate an acoustic impedance of a substance proximate the portion of platen. In some examples, an outer surface of the platen, or a layer residing on an outer surface of the platen, may have an acoustic impedance that is configured to approximate the acoustic impedance of human skin. In some examples, the apparatus may include one or more mirror layers configured to reflect light away from one or more portions of the ultrasonic receiver system.

In some examples, the apparatus may include a layer residing between the platen and the one or more receiver elements. An acoustic impedance of the layer may be in an acoustic impedance range between an acoustic impedance of the platen and an acoustic impedance of the one or more receiver elements.

According to some examples, the apparatus may include one or more anti-reflective layers. In some such examples, at least one of the one or more anti-reflective layers may reside proximate the outer surface of the platen. As used herein, “anti-reflective” refers to light. In other words, an anti-reflective layer is a layer that is configured to reduce or suppress the reflection of light.

In some examples, at least a portion of the platen may include an acoustic lens system. In some such examples, the acoustic lens system may include a spherical lens, a cylindrical lens, or both. According to some examples, the acoustic lens system may reside on, or proximate, an outer surface of the platen.

According to some examples, the apparatus may include one or more optical waveguides. In some such examples, at least a portion of one of the one or more optical waveguides may reside in a portion of the platen.

In some implementations, the apparatus may include a control system. The control system may include one or more general purpose single-or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. According to some examples, the control system may be configured to control the light source system to emit light and to receive signals from the ultrasonic receiver corresponding to the ultrasonic waves generated by the target object. In some such examples, the control system may be configured to identify blood vessel signals from the ultrasonic receiver corresponding to ultrasonic waves generated by blood within a blood vessel of the target object, generated by one or more blood vessel walls, or a combination thereof. In some such examples, the control system may be configured to estimate one or more cardiac features based, at least in part, on the blood vessel signals.

Other innovative aspects of the subject matter described in this disclosure can be implemented in a method. The method may involve controlling, by the control system, the light source system to emit light. The method may involve receiving, by the control system, signals from the ultrasonic receiver corresponding to the ultrasonic waves generated by the target object. The method may involve identifying, by the control system, blood vessel signals from the ultrasonic receiver corresponding to ultrasonic waves generated by blood within a blood vessel of the target object, generated by one or more blood vessel walls, or a combination thereof. In some such examples, the method may involve estimating, by the control system, one or more cardiac features based, at least in part, on the blood vessel signals.

Some or all of the methods described herein may be performed by one or more devices according to instructions (e.g., software) stored on non-transitory media. Such non-transitory media may include memory devices such as those described herein, including but not limited to random access memory (RAM) devices, read-only memory (ROM) devices, etc. Accordingly, some innovative aspects of the subject matter described in this disclosure can be implemented in one or more non-transitory media having software stored thereon. The software may include instructions for controlling one or more devices to perform one or more disclosed methods.

According to some such examples, the method may involve controlling, by the control system, the light source system to emit light. The method may involve receiving, by the control system, signals from the ultrasonic receiver corresponding to the ultrasonic waves generated by the target object. The method may involve identifying, by the control system, blood vessel signals from the ultrasonic receiver corresponding to ultrasonic waves generated by blood within a blood vessel of the target object, generated by one or more blood vessel walls, or a combination thereof. In some such examples, the method may involve estimating, by the control system, one or more cardiac features based, at least in part, on the blood vessel signals.

Details of one or more implementations of the subject matter described in this disclosure are set forth in the accompanying drawings and the description below. Other features, aspects, and advantages will become apparent from the description, the drawings and the claims. Note that the relative dimensions of the following figures may not be drawn to scale.

### BRIEF DESCRIPTION OF THE DRAWINGS

FIG. 1 shows an example of a blood pressure monitoring device based on photoplethysmography (PPG).

FIG. 2 shows an example of a blood pressure monitoring device based on photoacoustic plethysmography, which may be referred to herein as PAPG.

FIG. 3 is a block diagram that shows example components of an apparatus according to some disclosed implementations.

FIG. 4A shows example components of an apparatus according to some disclosed implementations.

FIG. 4B shows example components of an apparatus according to some disclosed implementations.

FIG. 5 shows example components of an alternative apparatus according to some disclosed implementations.

FIG. 6 shows example components of an alternative apparatus according to some additional disclosed implementations.

FIG. 7 shows example components of an alternative apparatus according to some disclosed implementations.

FIGS. 8, 9 and 10 show example components of devices according to some alternative implementations.

FIG. 11 shows example components of an apparatus according to some additional implementations.

FIG. 12 shows example components of an apparatus according to some additional implementations.

FIG. 13 shows example components of an apparatus according to some additional implementations.

FIG. 14 shows example components of an apparatus according to some additional implementations.

FIG. 15 shows example components of an apparatus according to some additional implementations.

FIG. 16 shows example components of an apparatus according to some additional implementations.

FIG. 17 shows example components of an apparatus according to some additional implementations.

FIG. 18 shows example components of an apparatus according to some additional implementations.

FIG. 19 shows example components of an apparatus according to some additional implementations.

FIG. 20 shows example components of an apparatus portion according to some additional implementations.

FIG. 21 shows example components of an apparatus portion according to some implementations.

FIG. 22 shows example components of an apparatus portion according to some alternative implementations.

FIG. 23 is a flow diagram that shows examples of some disclosed operations.

FIG. 24 shows examples of heart rate waveform (HRW) features that may be extracted according to some implementations of the method of FIG. 23.

FIG. 25 shows examples of devices that may be used in a system for estimating blood pressure based, at least in part, on pulse transit time (PTT).

FIG. 26 shows a cross-sectional side view of a diagrammatic representation of a portion of an artery through which a pulse is propagating.

FIG. 27A shows an example ambulatory monitoring device designed to be worn around a wrist according to some implementations.

FIG. 27B shows an example ambulatory monitoring device designed to be worn on a finger according to some implementations.

FIG. 27C shows an example ambulatory monitoring device designed to reside on an earbud according to some implementations.

Like reference numbers and designations in the various drawings indicate like elements.

### DETAILED DESCRIPTION

The following description is directed to certain implementations for the purposes of describing various aspects of this disclosure. However, a person having ordinary skill in the art will readily recognize that the teachings herein can be applied in a multitude of different ways. Some of the concepts and examples provided in this disclosure are especially applicable to blood pressure monitoring applications. However, some implementations also may be applicable to other types of biological sensing applications, as well as to other fluid flow systems. The described implementations may be implemented in any device, apparatus, or system that includes an apparatus as disclosed herein. In addition, it is contemplated that the described implementations may be included in or associated with a variety of electronic devices such as, but not limited to: mobile telephones, multimedia Internet enabled cellular telephones, mobile television receivers, wireless devices, smartphones, smart cards, wearable devices such as bracelets, armbands, wristbands, rings, headbands, patches, etc., Bluetooth® devices, personal data assistants (PDAs), wireless electronic mail receivers, hand-held or portable computers, netbooks, notebooks, smartbooks, tablets, printers, copiers, scanners, facsimile devices, global positioning system (GPS) receivers/navigators, cameras, digital media players, game consoles, wrist watches, clocks, calculators, television monitors, flat panel displays, electronic reading devices (e.g., e-readers), mobile health devices, computer monitors, auto displays (including odometer and speedometer displays, etc.), cockpit controls and/or displays, camera view displays (such as the display of a rear view camera in a vehicle), architectural structures, microwaves, refrigerators, stereo systems, cassette recorders or players, DVD players, CD players, VCRs, radios, portable memory chips, washers, dryers, washer/dryers, parking meters, automobile doors, autonomous or semi-autonomous vehicles, drones, Internet of Things (IoT) devices, etc. Thus, the teachings are not intended to be limited to the specific implementations depicted and described with reference to the drawings; rather, the teachings have wide applicability as will be readily apparent to persons having ordinary skill in the art.

Non-invasive health monitoring devices, such as photoacoustic plethysmography (PAPG)-based devices, have various potential advantages over more invasive health monitoring devices such as cuff-based or catheter-based blood pressure measurement devices. However, it has proven to be difficult to design satisfactory compact, or semi-compact, PAPG-based devices. (Some “semi-compact” devices may have a length in the range of 5.0 mm to 40 mm. Some semi-compact devices may have a cross-sectional area in the range of 6.0 mm² to 50 mm². A “compact” device is a device that is smaller than a semi-compact device.) For example, some semi-compact devices that have recently been developed by the present assignee to mitigate artifact signals such as electromagnetic interference (EMI) signals, signals from reflected light and signals from reflected acoustic waves, may be too large to deploy conveniently in a wearable device, such as a watch, a patch or an ear bud.

Some disclosed devices include a platen, a light source system and an ultrasonic receiver system. According to some implementations, the light source system may be configured to direct light along a first axis oriented at a first angle relative to an outer surface of the platen on which a target object may be placed. In some such implementations, the normal to the receiver plane may be oriented along a second axis at a second angle relative to the outer surface of the platen. In some implementations, the platen may include an anti-reflective layer, a mirror layer, or combinations thereof. According to some implementations, the platen may have an outer surface, or a layer on the outer surface, with an acoustic impedance that is configured to approximate the acoustic impedance of human skin. In some implementations, the platen may have an surface proximate the ultrasonic receiver system, or a layer on the surface proximate the ultrasonic receiver system, with an acoustic impedance that is configured to approximate the acoustic impedance of the ultrasonic receiver system.

Particular implementations of the subject matter described in this disclosure can be implemented to realize one or more of the following potential advantages. Various disclosed configurations include PAPG-capable devices that are compact enough to reside in a wearable device. In some implementations that include an anti-reflective layer, a mirror layer, or combinations thereof, such elements may be configured to reduce the amount of light from the light source system that is received by the ultrasonic receiver system, thereby mitigating noise. Acoustic impedance matching layers may mitigate unwanted reflections of acoustic waves, thereby mitigating another type of noise.

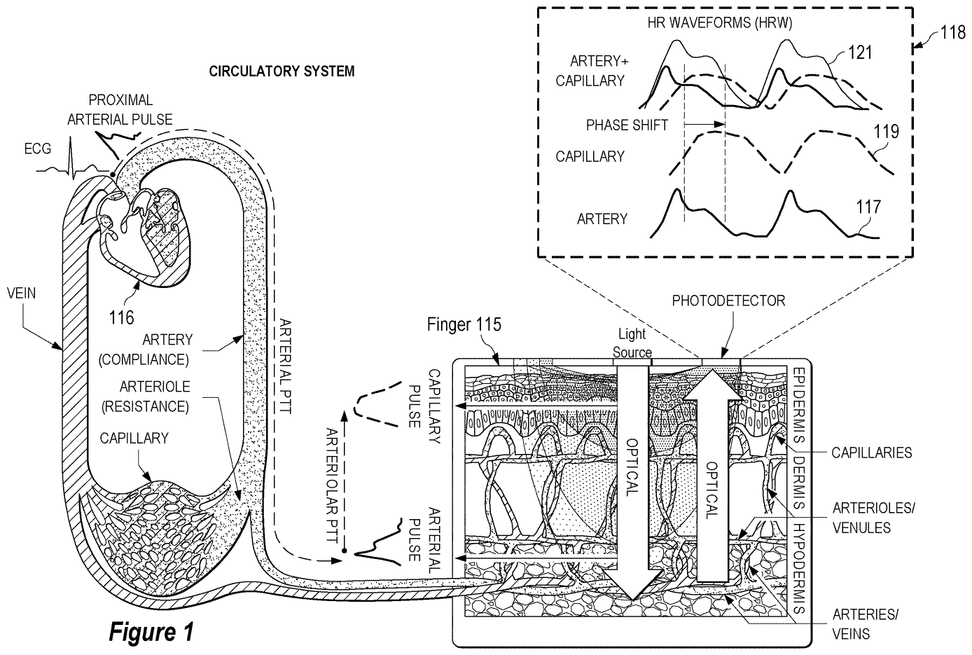

FIG. 1 shows an example of a blood pressure monitoring device based on photoplethysmography (PPG). FIG. 1 shows examples of arteries, veins, arterioles, venules and capillaries of a circulatory system, including those inside a finger 115. In the example shown in FIG. 1, an electrocardiogram (ECG) sensor has detected a proximal arterial pulse near the heart 116. Some examples are described below of measurement of the arterial pulse transit time (PTT) according to arterial pulses measured by two sensors, one of which may be an electrocardiogram sensor in some implementations.

According to the example shown in FIG. 1, a light source that includes one or more lasers or light-emitting diodes (LEDs) has transmitted light (in some examples, green, red, and/or near-infrared (NIR) light) that has penetrated the tissues of the finger 115 in an illuminated zone. Reflections from these tissues, detected by the photodetector, may be used to detect volumetric changes in the blood of the illuminated zone of the finger 115 that correspond to heart rate waveforms.

As shown in the heart rate waveform graphs 118 of FIG. 1, the capillary heart rate waveform 119 is differently-shaped and phase-shifted relative to the artery heart rate waveform 117. In this simple example, the detected heart rate waveform 121 is a combination of the capillary heart rate waveform 119 and the artery heart rate waveform 117. In some instances, the responses of one or more other blood vessels may also be part of the heart rate waveform 121 detected by a PPG-based blood pressure monitoring device. PPG-based blood pressure monitoring devices are not optimal because PPG superimposes data corresponding to the blood volume of all illuminated blood vessels, each of which exhibit different and time-shifted blood volume changes. Nonetheless, there are many deployed PPG-based blood pressure monitoring devices.

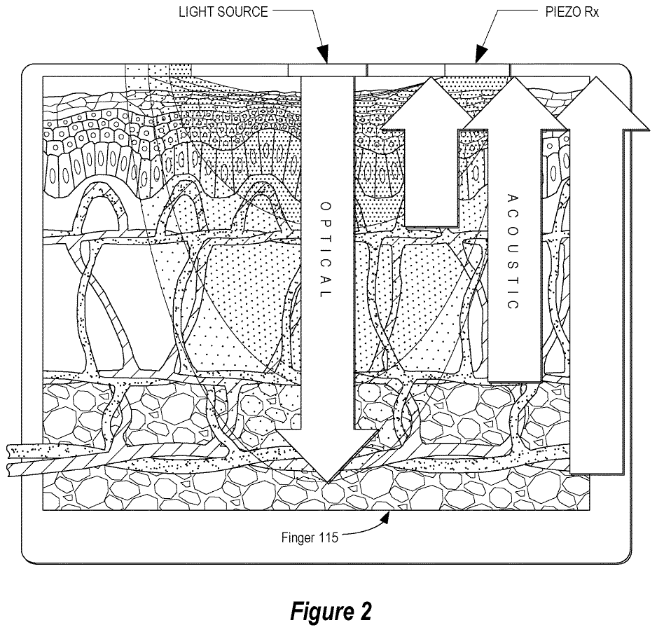

FIG. 2 shows an example of a blood pressure monitoring device based on photoacoustic plethysmography, which may be referred to herein as PAPG. FIG. 2 shows the same examples of arteries, veins, arterioles, venules and capillaries inside the finger 115 that are shown in FIG. 1. In some examples, the light source shown in FIG. 2 may be, or may include, one or more LEDs, one or more laser diodes, etc. In this example, as in FIG. 1, the light source has transmitted light (in some examples, green, red, and/or near-infrared (NIR) light) that has penetrated the tissues of the finger 115 in an illuminated zone.

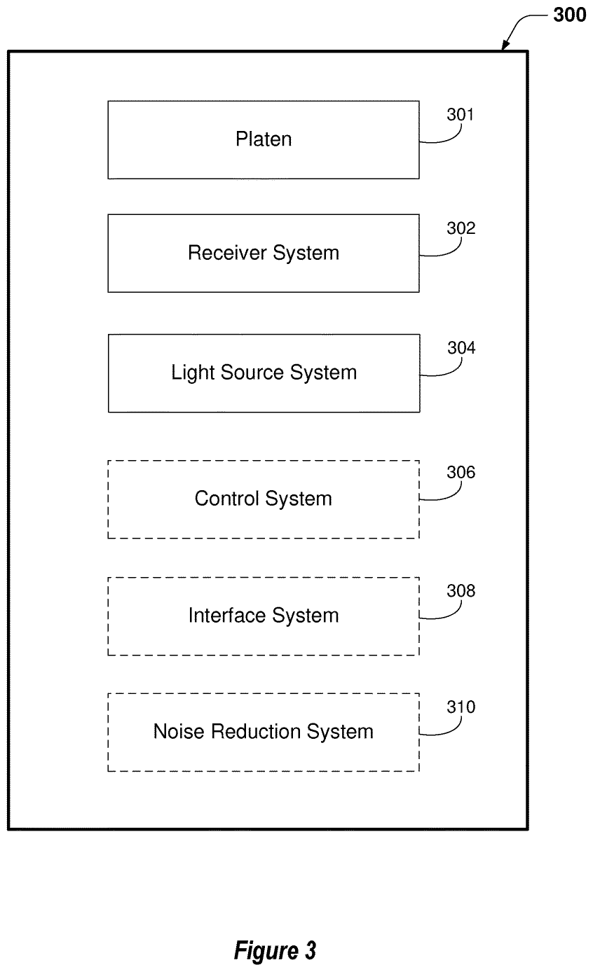

In the example shown in FIG. 2, blood vessels (and components of the blood itself) are heated by the incident light from the light source and are emitting acoustic waves. In this example, the emitted acoustic waves include ultrasonic waves. According to this implementation, the acoustic wave emissions are being detected by an ultrasonic receiver, which is a piezoelectric receiver in this example. Photoacoustic emissions from the illuminated tissues, detected by the piezoelectric receiver, may be used to detect volumetric changes in the blood of the illuminated zone of the finger 115 that correspond to heart rate waveforms. Although some of the tissue areas shown to be illuminated are offset from those shown to be producing photoacoustic emissions, this is merely for illustrative convenience. It will be appreciated that that the illuminated tissues will actually be those producing photoacoustic emissions. Moreover, it will be appreciated that the maximum levels of photoacoustic emissions will often be produced along the same axis as the maximum levels of illumination. In some examples, the ultrasonic receiver may be an instance of the receiver system 302 that is described below with reference to FIG. 3.

One important difference between the PPG-based system of FIG. 1 and the PAPG-based method of FIG. 2 is that the acoustic waves shown in FIG. 2 travel much more slowly than the reflected light waves shown in FIG. 1. Accordingly, depth discrimination based on the arrival times of the acoustic waves shown in FIG. 2 is possible, whereas depth discrimination based on the arrival times of the light waves shown in FIG. 1 may not be possible. This depth discrimination allows some disclosed implementations to isolate acoustic waves received from the different blood vessels.

According to some such examples, such depth discrimination allows artery heart rate waveforms to be distinguished from vein heart rate waveforms and other heart rate waveforms. Therefore, blood pressure estimation based on depth-discriminated PAPG methods can be substantially more accurate than blood pressure estimation based on PPG-based methods.

FIG. 3 is a block diagram that shows example components of an apparatus according to some disclosed implementations. In this example, the apparatus 300 includes a platen 301, a receiver system 302 and a light source system 304. Some implementations of the apparatus 300 may include a control system 306, an interface system 308, a noise reduction system 310, or combinations thereof.

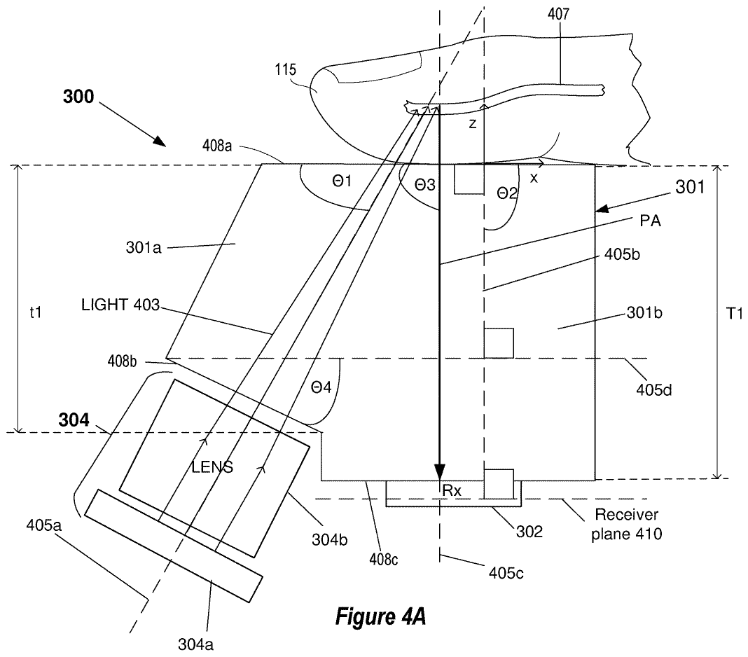

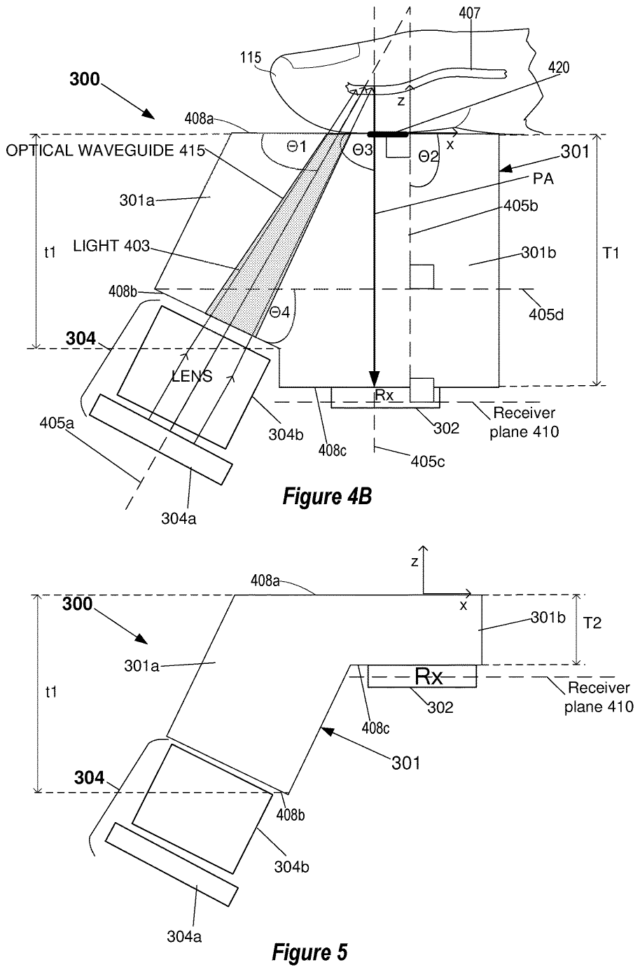

Various examples of platens 301 and various configurations of light source systems 304 and receiver systems 302 are disclosed herein. Some examples are described in more detail below with reference to FIGS. 4-19.

In some implementations in which the receiver system 302 includes an ultrasonic receiver system, the platen 301 may be configured to increase an intensity of ultrasonic energy received by at least a portion of the ultrasonic receiver system. In some such implementations, the platen 301 may include an acoustic waveguide. According to some implementations, the platen 301 may include an acoustic lens system. The acoustic lens system may, for example, reside on, or proximate, an outer surface of the platen 301. The acoustic lens system may, for example, include a spherical lens or a cylindrical lens. In some such examples, an outer surface of the platen 301 may include a recess having a shape corresponding to a portion of a sphere, a portion of a cylinder, or both. According to some examples, at least a portion of the ultrasonic receiver system may provide acoustic focusing functionality. For example, at least a portion of the ultrasonic receiver system may reside on a curved surface.

According to some examples, the platen 301, the light source system 304, or a combination thereof, may be configured for transmitting at least some of the light from the light source system to an outer surface of the platen (or to a target object on, or proximate, the outer surface) along a first axis, or substantially along the first axis oriented at a first angle relative to the outer surface of the platen. In this context, “substantially along the first axis” may mean within an angle range of plus or minus 10 degrees of the first axis, within an angle range of plus or minus 15 degrees of the first axis, within an angle range of plus or minus 20 degrees of the first axis, within an angle range of plus or minus 25 degrees of the first axis, within an angle range of plus or minus 30 degrees of the first axis, or within another such angle range.

In some examples, the light source system 304 may include a light source system surface having a normal that is parallel, or substantially parallel, to the first axis. In some such examples, a light source of the light source system may reside on, or proximate, the light source system surface.

According to some examples, the receiver system 302 may include one or more receiver elements residing in a receiver plane. A normal to the receiver plane may be oriented along a second axis, or substantially along the second axis, at a second angle relative to the outer surface of the platen. In this context, “substantially along the second axis” may mean within an angle range of plus or minus 10 degrees of the second axis, within an angle range of plus or minus 15 degrees of the second axis, within an angle range of plus or minus 20 degrees of the second axis, within an angle range of plus or minus 25 degrees of the second axis, within an angle range of plus or minus 30 degrees of the second axis, or within another such angle range. According to some examples, the second axis may be parallel to the first axis. However, in some examples, the second angle may be different from the first angle.

In some examples, at least a portion of the platen 301 may be configured for transmitting at least some of the ultrasonic waves generated by the target object along a third axis, or substantially along the third axis, that is at a third angle relative to the outer surface of the platen. According to some examples, the third axis may be parallel to the second axis.

According to some examples, the platen 301 may include different portions, which may have varying thicknesses, orientations, etc., depending on the particular implementation. In some examples, the platen 301 may include a first platen portion residing between the light source system and the outer surface of the platen. In some examples, the first platen portion may have a first platen portion thickness that is less than a second platen portion thickness of a second platen portion residing between at least one receiver element of the receiver system 302 and the outer surface of the platen. In some such examples, the first platen portion may be configured to receive light from the light source system and may also be configured to reflect the ultrasonic waves generated by the target object towards at least one receiver element of the receiver system. However, in some examples, a first platen portion residing between the light source system and the outer surface of the platen may have a first platen portion thickness that is greater than a second platen portion residing between at least one receiver element of the receiver system 302 and the outer surface of the platen.

According to some examples, the platen 301 (or another portion of the apparatus) may include one or more anti-reflective layers. In some examples, one or more anti-reflective layers may reside on, or proximate, one or more outer surfaces of the platen 301.

In some examples, at least a portion of the outer surface of the platen 301 may have an acoustic impedance that is configured to approximate an acoustic impedance of human skin. The portion of the outer surface of the platen 301 may, for example, be a portion that is configured to receive a target object, such as a human digit. (As used herein, the terms “finger” and “digit” may be used interchangeably, such that a thumb is one example of a finger.) A typical range of acoustic impedances for human skin is 1.53-1.680 MRayls. In some examples, at least an outer surface of the platen 301 may have an acoustic impedance that is in the range of 1.4-1.8 MRayls, or in the range of 1.5-1.7 MRayls.

Alternatively, or additionally, in some examples at least an outer surface of the platen 301 may be configured to conform to a surface of human skin. In some such examples, at least an outer surface of the platen 301 may have material properties like those of putty or chewing gum.

In some examples, at least a portion of the platen 301 may have an acoustic impedance that is configured to approximate an acoustic impedance of one or more receiver elements of the receiver system 302. According to some examples, a layer residing between the platen 301 and one or more receiver elements may have an acoustic impedance that is configured to approximate an acoustic impedance of the one or more receiver elements. Alternatively, or additionally, in some examples a layer residing between the platen 301 and one or more receiver elements may have an acoustic impedance that is in an acoustic impedance range between an acoustic impedance of the platen and an acoustic impedance of the one or more receiver elements.

Various examples of receiver systems 302 are disclosed herein, some of which may include ultrasonic receiver systems, optical receiver systems, or combinations thereof. In some implementations that include an ultrasonic receiver system, the ultrasonic receiver and an ultrasonic transmitter may be combined in an ultrasonic transceiver. In some examples, the receiver system 302 may include a piezoelectric receiver layer, such as a layer of PVDF polymer or a layer of PVDF-TrFE copolymer. In some implementations, a single piezoelectric layer may serve as an ultrasonic receiver. In some implementations, other piezoelectric materials may be used in the piezoelectric layer, such as aluminum nitride (AlN) or lead zirconate titanate (PZT). The receiver system 302 may, in some examples, include an array of ultrasonic transducer elements, such as an array of piezoelectric micromachined ultrasonic transducers (PMUTs), an array of capacitive micromachined ultrasonic transducers (CMUTs), etc. In some such examples, a piezoelectric receiver layer, PMUT elements in a single-layer array of PMUTs, or CMUT elements in a single-layer array of CMUTs, may be used as ultrasonic transmitters as well as ultrasonic receivers. According to some examples, the receiver system 302 may be, or may include, an ultrasonic receiver array. In some examples, the apparatus 300 may include one or more separate ultrasonic transmitter elements. In some such examples, the ultrasonic transmitter(s) may include an ultrasonic plane-wave generator.

The light source system 304 may, in some examples, include one or more light-emitting diodes. In some implementations, the light source system 304 may include one or more laser diodes. According to some implementations, the light source system 304 may include one or more vertical-cavity surface-emitting lasers (VCSELs). In some implementations, the light source system 304 may include one or more edge-emitting lasers. In some implementations, the light source system may include one or more neodymium-doped yttrium aluminum garnet (Nd:YAG) lasers.

According to some examples, the light source system 304 may include one or more light-directing elements configured to direct light from the light source system towards the target object along the first axis. In some examples, the one or more light-directing elements may include at least one diffraction grating. Alternatively, or additionally, the one or more light-directing elements may include at least one lens.

The light source system 304 may, in some examples, be configured to transmit light in one or more wavelength ranges. In some examples, the light source system 304 may configured for transmitting light in a wavelength range of 500 to 600 nanometers (nm). According to some examples, the light source system 304 may configured for transmitting light in a wavelength range of 800 to 950 nm. In view of factors such as skin reflectance, fluence, the absorption coefficients of blood and various tissues, and skin safety limits, one or both of these wavelength ranges may be suitable for various use cases. For example, the wavelength ranges of 500 nm to 600 nm and of 800 to 950 nm may both be suitable for obtaining photoacoustic responses from relatively smaller, shallower blood vessels, such as blood vessels having diameters of approximately 0.5 mm and depths in the range of 0.5 mm to 1.5 mm, such as may be found in a finger. The wavelength range of 800 to 950 nm may, for example, be suitable for obtaining photoacoustic responses from relatively larger, deeper blood vessels, such as blood vessels having diameters of approximately 2.0 mm and depths in the range of 2 mm to 3 mm, such as may be found in an adult wrist.

The light source system 304 may include various types of drive circuitry, depending on the particular implementation. In some disclosed implementations, the light source system 304 may include at least one multi-junction laser diode, which may produce less noise than single-junction laser diodes. In some examples, the light source system 304 may include a drive circuit (also referred to herein as drive circuitry) configured to cause the light source system to emit pulses of light at pulse widths in a range from 3 nanoseconds to 1000 nanoseconds. According to some examples, the light source system 304 may include a drive circuit configured to cause the light source system to emit pulses of light at pulse repetition frequencies in a range from 1 kilohertz to 100 kilohertz.

In some implementations, the apparatus (for example, the receiver system 302, the light source system 304, or both) may include one or more sound-absorbing layers, acoustic isolation material, light-absorbing material, light-reflecting material, or combinations thereof. In some examples, acoustic isolation material may reside between the light source system 304 and at least a portion of the receiver system 302. In some examples, the apparatus (for example, the receiver system 302, the light source system 304, or both) may include one or more electromagnetically shielded transmission wires. In some such examples, the one or more electromagnetically shielded transmission wires may be configured to reduce electromagnetic interference from the light source system 304 that is received by the receiver system 302.

In some implementations, the light source system 304 may be configured for emitting various wavelengths of light, which may be selectable to trigger acoustic wave emissions primarily from a particular type of material. For example, because the hemoglobin in blood absorbs near-infrared light very strongly, in some implementations the light source system 304 may be configured for emitting one or more wavelengths of light in the near-infrared range, in order to trigger acoustic wave emissions from hemoglobin. However, in some examples the control system 306 may control the wavelength(s) of light emitted by the light source system 304 to preferentially induce acoustic waves in blood vessels, other soft tissue, and/or bones. For example, an infrared (IR) light-emitting diode LED may be selected and a short pulse of IR light emitted to illuminate a portion of a target object and generate acoustic wave emissions that are then detected by the receiver system 302. In another example, an IR LED and a red LED or other color such as green, blue, white or ultraviolet (UV) may be selected and a short pulse of light emitted from each light source in turn with ultrasonic images obtained after light has been emitted from each light source. In other implementations, one or more light sources of different wavelengths may be fired in turn or simultaneously to generate acoustic emissions that may be detected by the ultrasonic receiver. Image data from the ultrasonic receiver that is obtained with light sources of different wavelengths and at different depths (e.g., varying RGDs) into the target object may be combined to determine the location and type of material in the target object. Image contrast may occur as materials in the body generally absorb light at different wavelengths differently. As materials in the body absorb light at a specific wavelength, they may heat differentially and generate acoustic wave emissions with sufficiently short pulses of light having sufficient intensities. Depth contrast may be obtained with light of different wavelengths and/or intensities at each selected wavelength. That is, successive images may be obtained at a fixed RGD (which may correspond with a fixed depth into the target object) with varying light intensities and wavelengths to detect materials and their locations within a target object. For example, hemoglobin, blood glucose or blood oxygen within a blood vessel inside a target object such as a finger may be detected photoacoustically.

According to some implementations, the light source system 304 may be configured for emitting a light pulse with a pulse width less than about 100 nanoseconds. In some implementations, the light pulse may have a pulse width between about 10 nanoseconds and about 500 nanoseconds or more. According to some examples, the light source system may be configured for emitting a plurality of light pulses at a pulse repetition frequency between 10 Hz and 100 kHz. Alternatively, or additionally, in some implementations the light source system 304 may be configured for emitting a plurality of light pulses at a pulse repetition frequency between about 1 MHz and about 100 MHz. Alternatively, or additionally, in some implementations the light source system 304 may be configured for emitting a plurality of light pulses at a pulse repetition frequency between about 10 Hz and about 1 MHz. In some examples, the pulse repetition frequency of the light pulses may correspond to an acoustic resonant frequency of the ultrasonic receiver and the substrate. For example, a set of four or more light pulses may be emitted from the light source system 304 at a frequency that corresponds with the resonant frequency of a resonant acoustic cavity in the sensor stack, allowing a build-up of the received ultrasonic waves and a higher resultant signal strength. In some implementations, filtered light or light sources with specific wavelengths for detecting selected materials may be included with the light source system 304. In some implementations, the light source system may contain light sources such as red, green and blue LEDs of a display that may be augmented with light sources of other wavelengths (such as IR and/or UV) and with light sources of higher optical power. For example, high-power laser diodes or electronic flash units (e.g., an LED or xenon flash unit) with or without filters may be used for short-term illumination of the target object.

The control system 306 may include one or more general purpose single-or multi-chip processors, digital signal processors (DSPs), application specific integrated circuits (ASICs), field programmable gate arrays (FPGAs) or other programmable logic devices, discrete gates or transistor logic, discrete hardware components, or combinations thereof. The control system 306 also may include (and/or be configured for communication with) one or more memory devices, such as one or more random access memory (RAM) devices, read-only memory (ROM) devices, etc. Accordingly, the apparatus 300 may have a memory system that includes one or more memory devices, though the memory system is not shown in FIG. 3. The control system 306 may be configured for receiving and processing data from the receiver system 302, e.g., as described below. If the apparatus 300 includes an ultrasonic transmitter, the control system 306 may be configured for controlling the ultrasonic transmitter. In some implementations, functionality of the control system 306 may be partitioned between one or more controllers or processors, such as a dedicated sensor controller and an applications processor of a mobile device.

In some examples, the control system 306 may be configured to control the light source system 304 to emit light towards a target object on an outer surface of the platen 301. In some such examples, the control system 306 may be configured to receive signals from the ultrasonic receiver system 302 corresponding to the ultrasonic waves generated by the target object responsive to the light from the light source system 304. In some examples, the control system 306 may be configured to identify one or more blood vessel signals, such as arterial signals or vein signals, from the ultrasonic receiver system. In some such examples, the one or more arterial signals or vein signals may be, or may include, one or more blood vessel wall signals corresponding to ultrasonic waves generated by one or more arterial walls or vein walls of the target object. In some such examples, the one or more arterial signals or vein signals may be, or may include, one or more arterial blood signals corresponding to ultrasonic waves generated by blood within an artery of the target object or one or more vein blood signals corresponding to ultrasonic waves generated by blood within a vein of the target object. In some examples, the control system 306 may be configured to estimate one or more cardiac features based, at least in part, on one or more arterial signals, on one or more vein signals, or on combinations thereof. According to some examples, the cardiac features may be, or may include, blood pressure.

Some implementations of the apparatus 300 may include the interface system 308. In some examples, the interface system 308 may include a wireless interface system. In some implementations, the interface system 308 may include a user interface system, one or more network interfaces, one or more interfaces between the control system 306 and a memory system and/or one or more interfaces between the control system 306 and one or more external device interfaces (e.g., ports or applications processors), or combinations thereof. According to some examples in which the interface system 308 is present and includes a user interface system, the user interface system may include a microphone system, a loudspeaker system, a haptic feedback system, a voice command system, one or more displays, or combinations thereof. According to some examples, the interface system 308 may include a touch sensor system, a gesture sensor system, or a combination thereof. The touch sensor system (if present) may be, or may include, a resistive touch sensor system, a surface capacitive touch sensor system, a projected capacitive touch sensor system, a surface acoustic wave touch sensor system, an infrared touch sensor system, any other suitable type of touch sensor system, or combinations thereof.

In some examples, the interface system 308 may include, a force sensor system. The force sensor system (if present) may be, or may include, a piezo-resistive sensor, a capacitive sensor, a thin film sensor (for example, a polymer-based thin film sensor), another type of suitable force sensor, or combinations thereof. If the force sensor system includes a piezo-resistive sensor, the piezo-resistive sensor may include silicon, metal, polysilicon, glass, or combinations thereof. An ultrasonic fingerprint sensor and a force sensor system may, in some implementations, be mechanically coupled. In some such examples, the force sensor system may be integrated into circuitry of the ultrasonic fingerprint sensor. In some examples, the interface system 308 may include an optical sensor system, one or more cameras, or a combination thereof.

According to some examples, the apparatus 300 may include a noise reduction system 310. For example, the noise reduction system 310 may include one or more mirrors that are configured to reflect light from the light source system 304 away from the receiver system 302. In some implementations, the noise reduction system 310 may include one or more sound-absorbing layers, acoustic isolation material, light-absorbing material, light-reflecting material, or combinations thereof. In some examples, the noise reduction system 310 may include acoustic isolation material, which may reside between the light source system 304 and at least a portion of the receiver system 302, on at least a portion of the receiver system 302, or combinations thereof. In some examples, the noise reduction system 310 may include one or more electromagnetically shielded transmission wires. In some such examples, the one or more electromagnetically shielded transmission wires may be configured to reduce electromagnetic interference from circuitry of the light source system 304, receiver system circuitry, or combinations thereof, that is received by the receiver system 302.

The apparatus 300 may be used in a variety of different contexts, many examples of which are disclosed herein. For example, in some implementations a mobile device may include the apparatus 300. In some such examples, the mobile device may be a smart phone. In some implementations, a wearable device may include the apparatus 300. The wearable device may, for example, be a bracelet, an armband, a wristband, a watch, a ring, a headband or a patch.

FIG. 4A shows example components of an apparatus according to some disclosed implementations. As with other figures provided herein, the numbers, types and arrangements of elements shown in FIG. 4A are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115, a wrist, etc.

According to this example, the light source system 304 includes a light-emitting portion 304a and a lens 304b. The light-emitting portion 304a may, for example, include one or more light-emitting diodes, one or more laser diodes, one or more VCSELs, one or more edge-emitting lasers, one or more neodymium-doped yttrium aluminum garnet (Nd:YAG) lasers, or combinations thereof. In this example, the lens 304b is configured to focus the light 403 emitted by the light-emitting portion 304a into a relatively smaller cross-sectional area, which increases the intensity of the light 403 received by a target object (such as the finger 115) on the outer surface 408a. Although FIG. 4A shows the light-emitting portion 304a and the lens 304b separated by a gap, the light-emitting portion 304a and the lens 304b will generally be positioned adjacent to one another.

In this example, the platen 301 includes platen portion 301a and platen portion 301b. According to this example, the platen portion 301a has a thickness of t1, which is less than the thickness T1 of the platen portion 301b. In this example, the platen portion 301a includes a surface 408b that is configured to receive the light 403 from the light source system 304. Although FIG. 4A shows the light source system 304 separated from the surface 408b by a gap, in some examples the light source system 304 will be positioned adjacent to the surface 408b, without a gap. According to this example, the platen portion 301a is configured to direct the light 403 from the light source system 304 towards the outer surface 408a and towards a target object, if any, on the outer surface 408a.

According to this example, the platen 301 (more specifically, the platen portion 301a) and the light source system 304 are configured for transmitting light 403 from the light source system 304 to the outer surface 408a of the platen 301 along a first axis, or substantially along a first axis, which is oriented at a first angle relative to the outer surface 408a. In FIG. 4A, the axis 405a is an example of the first axis and the angle Θ1 is an example of the first angle. In this context, “substantially along the first axis” or “substantially parallel to the first axis” may mean within an angle range of plus or minus 10 degrees of the first axis, within an angle range of plus or minus 15 degrees of the first axis, within an angle range of plus or minus 20 degrees of the first axis, within an angle range of plus or minus 25 degrees of the first axis, within an angle range of plus or minus 30 degrees of the first axis, or within another such angle range. According to this example, the axis 405a is perpendicular to the surface 408b.

In this example, the receiver system 302, which is an ultrasonic receiver system in this implementation, resides adjacent to a surface 408c of the platen 301 (more specifically, of the platen portion 301b). According to this example, the receiver system 302 resides in a receiver plane 410 that is oriented parallel to the surface 408c. In this example, a normal to the receiver plane 410 is oriented along a second axis, which in this example is the axis 405b, that is oriented at a second angle relative to the outer surface 408a. In FIG. 4A, the angle Θ2 is an example of the second angle. In this example, the receiver plane 410 is parallel to the outer surface 408a, so the angle Θ2 is 90 degrees.

According to this example, the platen 301 (more specifically, the platen portion 301b) is configured to direct acoustic waves, including photoacoustic waves PA, emitted by a target object on the outer surface 408a towards the receiver system 302. In this example, the platen 301 (more specifically, the platen portion 301b) is configured for transmitting acoustic waves, including but not limited to ultrasonic waves, generated by a target object on the outer surface 408a towards the receiver system 302 along a third axis, or substantially along the third axis, which is oriented at a third angle relative to the outer surface 408a. In FIG. 4A, the axis 405c is an example of the third axis and the angle Θ3 is an example of the third angle. In this context, “substantially along the third axis” or “substantially parallel to the third axis” may mean within an angle range of plus or minus 10 degrees of the third axis, within an angle range of plus or minus 15 degrees of the third axis, within an angle range of plus or minus 20 degrees of the third axis, within an angle range of plus or minus 25 degrees of the third axis, within an angle range of plus or minus 30 degrees of the third axis, or within another such angle range. According to this example, the platen portion 301b is shown to be transmitting arterial photoacoustic waves PA substantially along the axis 405c. In this example, the second axis is parallel to, or substantially parallel to the third axis (for example, within +/−5 degrees of being parallel, within +/−10 degrees of being parallel, within +/−15 degrees of being parallel,, within +/−20 degrees of being parallel, etc.).

In this example, the third axis is not parallel to the first axis, but instead is separated from the first axis by an angle (Θ3−Θ1). In some examples, the angle (Θ3−Θ1) may be in the range of 20 degrees to 60 degrees. According to some alternative examples, the third axis may be parallel to the first axis. However, in some alternative examples, the first axis may be parallel to, or substantially parallel to (for example, within +/−5 degrees of being parallel, within +/−10 degrees of being parallel, within +/−15 degrees of being parallel, within +/−20 degrees of being parallel, etc.) the third axis.

The first, second and third axes may, in some examples, be defined by a coordinate system that is relative to the apparatus 300 or a portion thereof. In the example shown in FIG. 4A, a Cartesian coordinate system is shown to be defined relative to the outer surface 408a of the platen 301.

In this example, an axis 405d is parallel to the outer surface 408a. An angle Θ4 is shown between the axis 405d and the surface 408b, indicating that the angle between the surface 408b and the outer surface 408a is also Θ4. According to this example, Θ4=Θ3−Θ1.

In some implementations, the platen 301 (for example, at least part of the platen portion 301b) may include an acoustic waveguide. In some such implementations, the platen portion 301b may be configured for transmitting ultrasonic waves generated by a target object on the outer surface 408a towards the receiver system 302, via the acoustic waveguide.

According to some examples, the platen 301 may include one or more anti-reflective layers. In some examples, one or more anti-reflective layers may reside on the platen 301, or proximate the platen 301, for example on or proximate the outer surface 408a.

FIG. 4B shows example components of an apparatus according to some disclosed implementations. As with other figures provided herein, the numbers, types and arrangements of elements shown in FIG. 4B are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115.

Many elements of the apparatus 300 shown in FIG. 4B are essentially the same as those shown in FIG. 4A. Therefore, the unchanged elements will not be described again here. In the example shown in FIG. 4B, the platen portion 301a includes an optical waveguide 415. In some instances, a hole may be formed in the platen portion 301a and an optical waveguide, which may include one or more optical fibers, may be inserted into the hole. According to some other examples, the platen portion 301a may be fabricated so as to include the optical waveguide 415. In some alternative examples, the optical waveguide 415 may be configured to direct light to an area of the outer surface 408a that is closer to a center portion of the receiver system 302, such as the area 420 shown in FIG. 4. Although other disclosed implementations may be shown without an optical waveguide 415, alternative versions of these disclosed implementations, including but not limited to those shown in FIGS. 4A and 5-19, may include one or more optical waveguides 415, or one or more of another type of optical waveguide.

FIG. 5 shows example components of an alternative apparatus according to some disclosed implementations. As with other figures provided herein, the numbers, types and arrangements of elements shown in FIG. 5 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as a finger, a wrist, etc.

According to this example, the light source system 304 includes a light-emitting portion 304a and a lens 304b. In this example, the lens 304b is configured to focus the light 403 emitted by the light-emitting portion 304a into a relatively smaller cross-sectional area, which increases the intensity of the light 403 received by a target object (such as the finger 115) on the outer surface 408a. Although FIG. 5 shows the light-emitting portion 304a and the lens 304b separated by a gap, the light-emitting portion 304a and the lens 304b will generally be positioned adjacent to one another.

In this example, the platen 301 includes platen portion 301a and platen portion 301b. In this example, the platen portion 301a includes a surface 408b that is configured to receive the light 403 from the light source system 304. Although FIG. 5 shows the light source system 304 separated from the surface 408b by a gap, in some examples the light source system 304 may be positioned adjacent to the surface 408b, without a gap. According to this example, the platen portion 301a is configured to direct the light 403 from the light source system 304 towards the outer surface 408a and towards a target object, if any, on the outer surface 408a.

According to this example, the platen portion 301a and the light source system 304 are configured for transmitting light 403 from the light source system 304 to the outer surface 408a of the platen 301 along a first axis, or substantially along a first axis, which is oriented at a first angle relative to the outer surface 408a. The first axis and the first angle may correspond to the axis 405a and the angle Θ1 shown in FIG. 4A.

In this example, the receiver system 302 resides adjacent to a surface 408c of the platen portion 301b in a receiver plane 410 that is oriented parallel to the surface 408c. In this example, a normal to the receiver plane 410 is oriented along a second axis, which is oriented at a second angle relative to the outer surface 408a. The second axis and the second angle may correspond to the axis 405b and the angle Θ2 shown in FIG. 4A. According to this example, the platen portion 301b is configured to direct acoustic waves, including photoacoustic waves PA, emitted by a target object on the outer surface 408a towards the receiver system 302 along a third axis, or substantially along the third axis, which is oriented at a third angle relative to the outer surface 408a. The third axis and the third angle may correspond to the axis 405c and the angle Θ3 shown in FIG. 4A.

In these examples, the relative orientations of the surfaces 408a, 408b and 408c, the first, second and third axes, the receiver plane 410 and the light source system 304 are as shown in FIG. 4A, with one exception: the platen portion 301b shown in FIG. 4A is substantially thicker than the platen portion 301b shown in FIG. 5. The platen portion 301b of FIG. 4A has a thickness of T1, which may be in the range of 7 mm to 15 mm in some examples, whereas the platen portion 301b shown in FIG. 5 has a thickness of T2, which may be in the range of 3 mm to 5 mm. According to this example, the platen portion 301a has a thickness of t1, which is greater than the thickness T2 of the platen portion 301b.

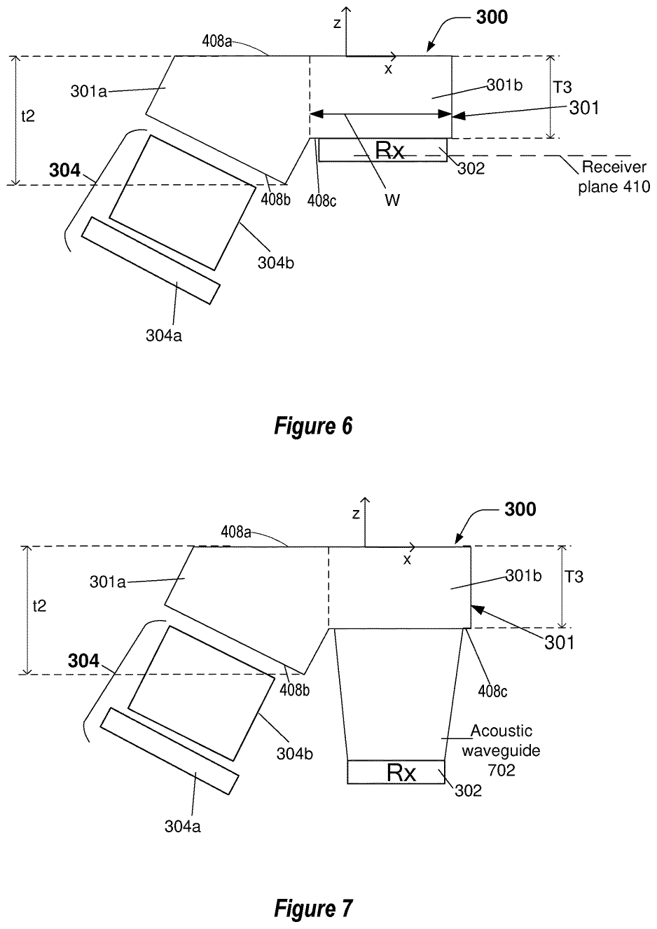

FIG. 6 shows example components of an alternative apparatus according to some additional disclosed implementations. As with other figures provided herein, the numbers, types and arrangements of elements shown in FIG. 6 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as a finger, a wrist, etc. According to this example, the platen portion 301a has a thickness of t2, which is greater than the thickness T3 of the platen portion 301b.

According to this example, the light source system 304 and the receiver system 302 are configured substantially as shown in FIGS. 4 and 5. In the examples shown in FIG. 6, the relative orientations of the surfaces 408a, 408b and 408c, the first, second and third axes, the receiver plane 410 and the light source system 304 are as described with reference to FIG. 5, with one exception: the platen portion 301a shown in FIG. 5 is substantially thicker than the platen portion 301a that is shown in FIG. 6. The platen portion 301a of FIG. 5 has a thickness of t1, which may be in the range of 7 mm to 15 mm in some examples, whereas the platen portion 301a shown in FIG. 6 has a thickness of t2, which may be in the range of 6 mm to 12 mm in some examples. In this example, the thickness T3 of the platen portion 301b is in the range of 3 mm to 7 mm, for example 5 mm. According to this example, the width W or the surface 408c is in the range of 5 mm to 10 mm, for example 7 or 8 mm.

FIG. 7 shows example components of an alternative apparatus according to some disclosed implementations. As with other figures provided herein, the numbers, types and arrangements of elements shown in FIG. 7 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as a finger, a wrist, etc. According to this example, the platen portion 301a has a thickness of t2, which is greater than the thickness T3 of the platen portion 301b.

According to this example, the light source system 304 and the receiver system 302 are configured substantially as shown in FIGS. 4-6. In the examples shown in FIG. 7, the relative orientations of the surfaces 408a, 408b and 408c, the first, second and third axes, the receiver plane 410 and the light source system 304 are as shown in FIG. 6, with one exception: in the example shown in FIG. 7, the apparatus 300 includes an acoustic waveguide 702 between the surface 408c and the receiver system 302.

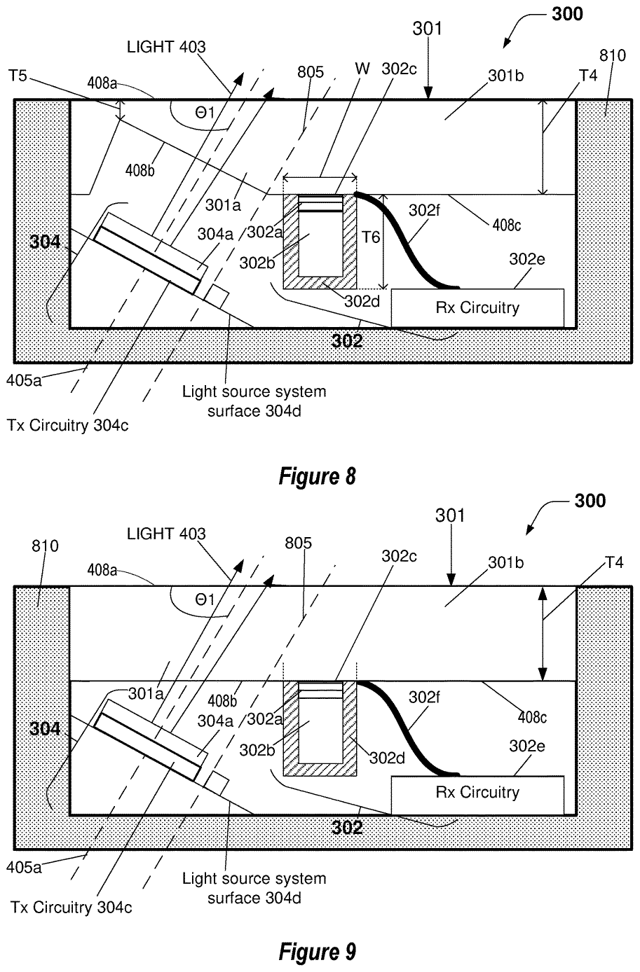

FIGS. 8, 9 and 10 show example components of devices according to some alternative implementations. As with other figures provided herein, the numbers, types and arrangements of elements shown in FIGS. 8-10 are merely presented by way of example. In each of these examples, the apparatus 300 is an instance of the apparatus 300 shown in FIG. 3. According to these examples, the apparatus 300 includes a platen 301, a receiver system 302 and a light source system 304. In these examples, an outer surface 408a of the platen 301 is configured to receive a target object, such as a finger, a wrist, etc.

According to the examples shown in FIGS. 8-10, the light source system 304 is not attached to the platen 301. Although not shown in FIG. 8 or FIG. 9, in some examples the light source system 304 may include a lens 304b. In the examples shown in FIGS. 8 and 9, the light-emitting portion 304a and the light source circuitry 304c both reside on a light source system surface 304d, a normal to which corresponds with the axis 805. In these examples, the axis 805 is parallel to, or substantially parallel to, the axis 405a. Although not shown in FIG. 8 or FIG. 9, in some examples the light source system 304 may include a lens 304b. In the examples shown in FIGS. 8 and 9, the light source system surface 304d is attached to the support structure 810 and is configured to cause the light-emitting portion 304a to emit at least some of the light 403 along the axis 405a, which is at an angle Θ1 relative to the outer surface 408a.

According to some examples, the support structure 810 may include acoustic isolation material, such as sound-absorbing material, that is configured for at least partially suppressing acoustic waves that may be generated by the light source circuitry 304c. In some such examples, at least a portion of the support structure 810 may be configured for at least partially de-coupling acoustic energy generated by the light source circuitry 304c from the platen 301 and the receiver system 302.

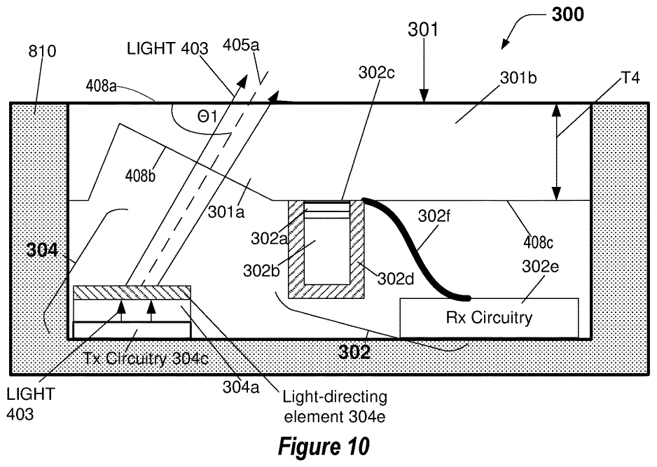

In the example shown in FIG. 10, the light-emitting portion 304a and the light source circuitry 304c both reside on a portion of the support structure 810 that is parallel to, or substantially parallel to, the outer surface 408a. Accordingly, much of the light 403 from the light-emitting portion 304a is emitted directly towards the outer surface 408a. However, in the example shown in FIG. 10, the light source system 304 includes a light-directing element 304e that is configured to re-direct at least some of the light 403 along the axis 405a. In some examples, the light-directing element 304e may be, or may include, a diffraction grating.

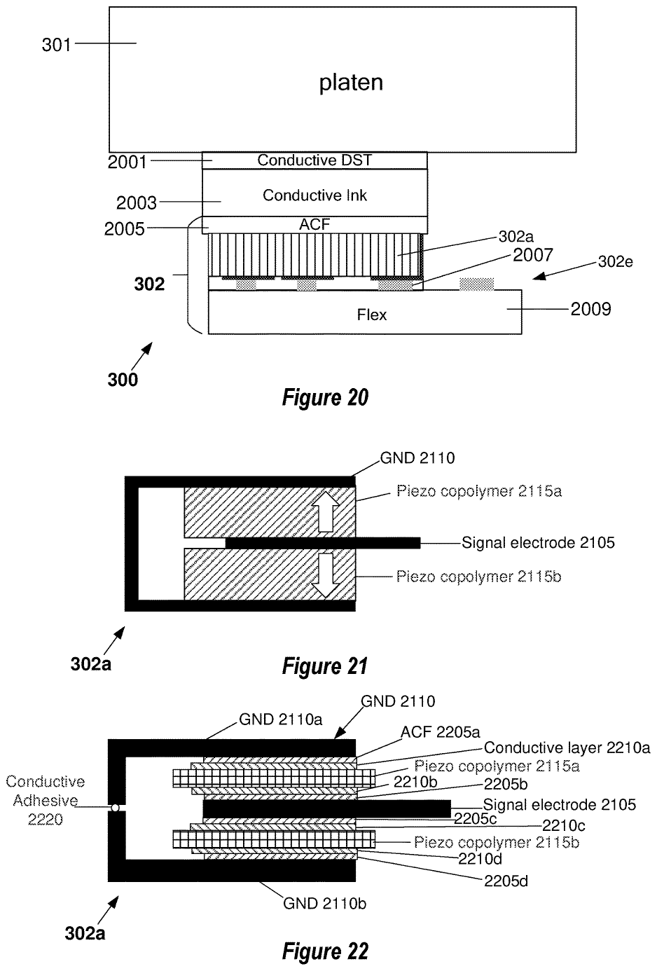

In the examples shown in FIGS. 8-10, the receiver system 302 includes a receiver portion 302a, a backer 302b, a mirror layer 302c, a noise-absorbing layer 302d, receiver system circuitry 302e and a connector 302f. The receiver portion 302a may include piezoelectric material, such as a piezoelectric copolymer, piezoelectric composite material, etc. Some detailed examples are described below with reference to FIGS. 20-22.

According to some examples, the noise-absorbing layer 302d may include light-blocking material (such as light-absorbing material), sound-absorbing material, or a combination thereof. In some examples, the noise-absorbing layer 302d may include an optically and acoustically isolating foam.

In these examples, the mirror layer 302c is configured to reflect light, including but not limited to light from the light source system 304 that is reflected from the outer surface 408a, away from the receiver portion 302a. According to some examples, anti-reflective material may reside on, or proximate, the outer surface 408a.

In the examples shown in FIGS. 8-10, the receiver system circuitry 302e resides on the support structure 810 and is not directly attached to the platen 301 or the receiver portion 302a. Such configurations can help to shield the receiver portion 302a from noise produced by the receiver system circuitry 302e. In these examples, connector 302f is configured for electrically connecting the receiver system circuitry 302e to the receiver portion 302a. According to some examples, the connector 302f may include electrically conductive material that is insulated by electromagnetic noise suppression material. Such examples can at least partially isolate the receiver portion 302a from electromagnetic noise produced by the receiver system circuitry 302e. The electromagnetic noise suppression material, the noise-absorbing layer 302d and the mirror layer 302c may be regarded as components of the noise reduction system 310 of FIG. 3.

According to these examples, the receiver system 302 includes a backer layer 302b. The backer layer 302b may be configured to suppress at least some acoustic artifacts and may provide a relatively higher signal-to-noise ratio (SNR) than receiver systems 302 that lack a backer layer. In these examples, the thickness T6 (shown in FIG. 8) of the receiver portion 302a, the backer 302b, the mirror layer 302c and the noise-absorbing layer 302d may be in the range of 5 mm to 10 mm, the majority of which (such as 5 mm to 7 mm) may correspond to the thickness of the backer layer 302b. Accordingly, including such a relatively thick backer layer 302b can enhance SNR, but may add to the overall device thickness of some implementations.

In the examples shown in FIGS. 8 and 10, the surface 408b of the platen 301 is parallel to, or substantially parallel to, the light source system surface 304d. In these examples, the surface 408b is perpendicular to, or substantially perpendicular to, the axis 405a. Accordingly, in these examples, the platen portion 301a has a thickness that ranges from T4 to T5, the latter of which may range from 1 mm to 3 mm. However, in the example shown in FIG. 9, the platen portions 301a and 301b each have a thickness of T4, which may be in the range from 3 mm to 7 mm in some instances.

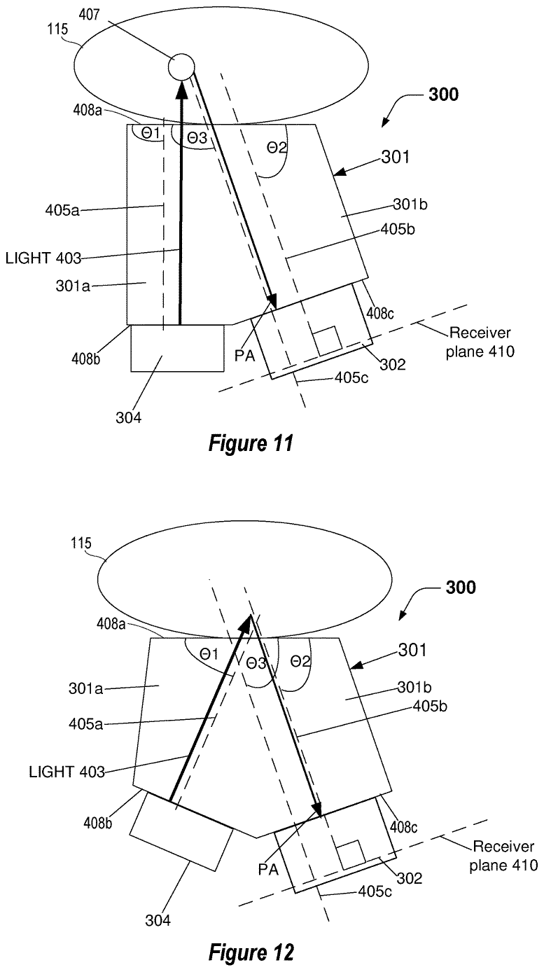

FIG. 11 shows example components of an apparatus according to some additional implementations. As with other figures provided herein, the numbers, types and arrangements of elements shown in FIG. 11 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115, a wrist, etc.

According to this example, the light source system 304 resides on a surface 408b of the platen 301 that is parallel to, or substantially parallel to, the outer surface 408a. Accordingly, in this example the angle Θ1, between the axis 405a and the outer surface 408a, is 90 degrees or approximately 90 degrees (such as within +/−10 degrees, within +/−15 degrees, within +/−20 degrees, etc.). In this example, the receiver system 302 resides on a surface 408c of the platen 301 that is not parallel to, or substantially parallel to, the outer surface 408a. Instead, the surface 408c and the receiver plane 410 are at an angle to the outer surface 408a such that the axis 405b (normal to the receiver plane 410) is oriented at an angle Θ2 with the outer surface 408a that is less than 90 degrees.

Such implementations have the potential advantage that much of the light 403 that is reflected from the outer surface 408a may be reflected back towards the light source system 304 instead of towards the receiver system 302. According to some examples, the platen 301 may include one or more anti-reflective layers in order to further mitigate such reflections. In some examples, one or more anti-reflective layers may reside on the platen 301, or proximate the platen 301, for example on or proximate the outer surface 408a.

One may observe that the implementation shown in FIG. 11 is similar to that shown in FIG. 4A, except that the receiver system 302 of FIG. 11 resides where the light source system 304 of FIG. 4A resides. Another difference is that in FIG. 11, the surfaces 408b and 408c are at an angle to one another, but are continuous: there is not a sudden decrease in thickness of the platen 301 from the surface 408c to the surface 408b, as shown in FIG. 4A, or vice versa.

FIG. 12 shows example components of an apparatus according to some additional implementations. As with other figures provided herein, the numbers, types and arrangements of elements shown in FIG. 12 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115, a wrist, etc.

One may observe that the implementation shown in FIG. 12 is similar to that shown in FIG. 11, except that in FIG. 12 the light source system 304 resides on a surface 408b of the platen 301 that is not parallel to, or substantially parallel to, the outer surface 408a. Instead, both angle Θ1 and angle Θ2 are less than 90 degrees. In some examples, angle Θ1 and angle Θ2 may be equal, or approximately equal (such as within +/−10 degrees, within +/−15 degrees, within +/−20 degrees, etc.).

In FIGS. 11 and 12, the outer surface 408a is shown to reside in one plane. In some alternative implementations, the outer surface 408a may reside in more than one plane. According to some such implementations, a portion of the outer surface 408a may reside in a plane that is parallel to, or substantially parallel to, the surface 408c of FIG. 11 or FIG. 12. Alternatively, or additionally, in some examples a portion of the outer surface 408a may reside in a plane that is parallel to, or substantially parallel to, the surface 408b of FIG. 12.

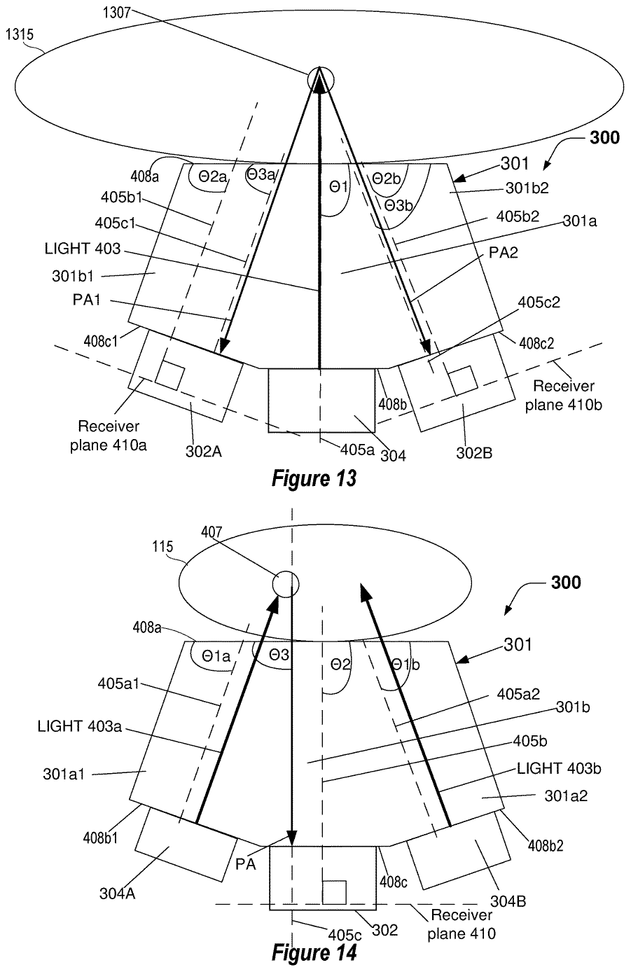

FIG. 13 shows example components of an apparatus according to some additional implementations. As with other figures provided herein, the numbers, types and arrangements of elements shown in FIG. 13 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the wrist 1315, a finger, etc.

According to this example, the light source system 304 resides on a surface 408b of the platen 301 that is parallel to, or substantially parallel to, the outer surface 408a. Accordingly, in this example the angle Θ1, between the axis 405a and the outer surface 408a, is 90 degrees or approximately 90 degrees (such as within +/−10 degrees, within +/−15 degrees, within +/−20 degrees, etc.). Here, the light source system 304 is configured to provide light to the outer surface 408a (and to a target object on the outer surface 408a, if any) via the platen portion 301a.

In this example, the receiver system 302 includes receiver system component 302A and receiver system component 302B. According to this example, the receiver system component 302A resides on a surface 408c1 of the platen 301 and the receiver system component 302B resides on a surface 408c2 of the platen 301. In this example, the surfaces 408c1 and 408c2 are not parallel to, or substantially parallel to, the outer surface 408a. Instead, the surface 408c1 and the corresponding receiver plane 410a are at an angle to the outer surface 408a such that the axis 405b1 (normal to the receiver plane 410a) is oriented at an angle Θ2a with the outer surface 408a that is less than 90 degrees. Similarly, the surface 408c2 and the corresponding receiver plane 410b are at an angle to the outer surface 408a such that the axis 405b2 (normal to the receiver plane 410b) is oriented at an angle Θ2b with the outer surface 408a that is less than 90 degrees. In some examples, the absolute value of the angle Θ2a may equal, or substantially equal, the absolute value of the angle Θ2b.

In this example, photoacoustic waves PA1 and PA2 are produced in the artery 1307 by the light 403. According to this example, at least a portion of the photoacoustic wave PA1 travels in the platen portion 301b1 along the axis 405c1, which is at an angle Θ3a with the outer surface 408a. In this example, at least a portion of the photoacoustic wave PA2 travels in the platen portion 301b2 along the axis 405c2, which is at an angle Θ3b with the outer surface 408a.

One may observe that the implementation shown in FIG. 13 is similar to that shown in FIG. 11, except that instead of having a single receiver system 302, as shown in FIG. 11, the implementation of FIG. 13 includes receiver system components 302A and 302B.

Implementations such as that shown in FIG. 13 have the potential advantage that much of the light 403 that is reflected from the outer surface 408a may be reflected back towards the light source system 304 instead of towards the receiver system component 302A or the receiver system component 302B. According to some examples, the platen 301 may include one or more anti-reflective layers in order to further mitigate such reflections. In some examples, one or more anti-reflective layers may reside on the platen 301, or proximate the platen 301, for example on or proximate the outer surface 408a. Another potential advantage of implementations like that shown in FIG. 13 is that more of the photoacoustic waves emitted by a target object may be captured because of the presence of multiple receiver system components, as compared to the single receiver system 302 shown in FIG. 11.

FIG. 14 shows example components of an apparatus according to some additional implementations. As with other figures provided herein, the numbers, types and arrangements of elements shown in FIG. 14 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115, a wrist, etc.

According to this example, the receiver system 302 resides on a surface 408c of the platen 301 that is parallel to, or substantially parallel to, the outer surface 408a. Accordingly, in this example the angle Θ2, between the axis 405b (normal to the receiver plane 410) and the outer surface 408a, is 90 degrees or approximately 90 degrees (such as within +/−10 degrees, within +/−15 degrees, within +/−20 degrees, etc.).

Here, the light source system 304 includes light source system component 304A and light source system component 304B. The light source system components 304A and 304B are configured to provide light 403a and 403b to the outer surface 408a (and to a target object on the outer surface 408a, if any) via the platen portions 301a1 and 301a2. According to this example, at least some of the light 403a travels substantially parallel to the axis 405a1, which is at an angle Θ1a with the outer surface 408a and at least some of the light 403b travels substantially parallel to the axis 405a2, which is at an angle Θ1b with the outer surface 408a. In some examples, the absolute value of the angle Θ1a may equal, or substantially equal, the absolute value of the angle Θ1b.

In this example, photoacoustic waves PA are produced in the blood vessel 407 by the light 403. According to this example, at least a portion of the photoacoustic waves PA travels in the platen portion 301b along the axis 405c, which is at an angle Θ3 with the outer surface 408a.

One may observe that the implementation shown in FIG. 14 is similar to that shown in FIG. 13, except that instead of having a single light source system 304 and two receiver system components, as shown in FIG. 13, the implementation of FIG. 14 includes a single receiver system 302 and light source system components 304A and 302B.

Implementations such as that shown in FIG. 14 have the potential advantage that additional light energy may be provided to a target object, as compared to implementations that include only a single light source system. Another potential advantage of implementations like that shown in FIG. 14 is that additional light paths into a target object may be provided. In the example shown in FIG. 14, for example, the path of the light 403a is more favorable for illuminating the blood vessel 407 than the path of the light 403b.

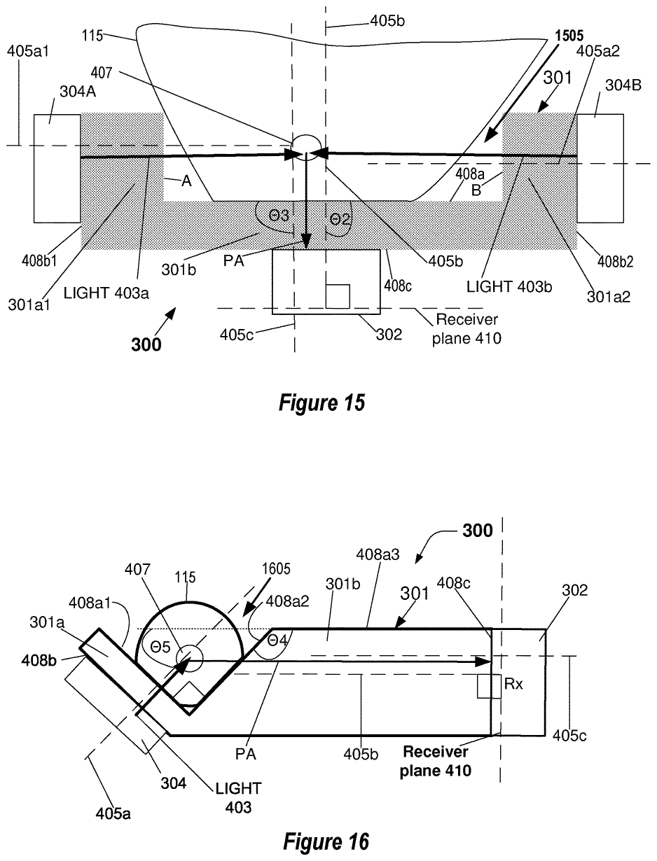

FIG. 15 shows example components of an apparatus according to some additional implementations. As with other figures provided herein, the numbers, types and arrangements of elements shown in FIG. 15 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115, etc.

According to this example, the receiver system 302 resides on a surface 408c of the platen 301 that is parallel to, or substantially parallel to, the outer surface 408a. Accordingly, in this example the angle Θ2, between the axis 405b (normal to the receiver plane 410) and the outer surface 408a, is 90 degrees or approximately 90 degrees (such as within +/−5 degrees, within +/−10 degrees, within +/−15 degrees, within +/−20 degrees, etc.).

Here, the light source system 304 includes light source system component 304A and light source system component 302B. In this example, the light source system component 304A is coupled to the surface 408b1 and is configured to provide light 403a to a target object on the outer surface 408a, if any, via the platen portion 301a1 and the outer surface A. In this example, the light source system component 304B is coupled to the surface 408b2 and is configured to provide light 403b to a target object on the outer surface 408a, if any, via the platen portion 301a2 and the outer surface B. In some examples, a target object may be in contact with the outer surface A, the outer surface B, or both.

According to this example, at least some of the light 403a travels substantially parallel to the axis 405a1, which is parallel to, or substantially parallel to, the outer surface 408a and perpendicular to, or substantially perpendicular to, the outer surface A. In this example, at least some of the light 403b travels substantially parallel to the axis 405a2, which is parallel to, or substantially parallel to, the outer surface 408a and perpendicular to, or substantially perpendicular to, the outer surface B. In this example, the light 403a travels in a direction that is opposite from the direction in which the light 403b travels.

In this example, photoacoustic waves PA are produced in the blood vessel 407 by the light 403a, the light 403b, or a combination thereof. According to this example, at least a portion of the photoacoustic waves PA travel in the platen portion 301b along the axis 405c, which is at an angle Θ3 with the outer surface 408a. In this example, the angle Θ3 is 90 degrees, or approximately 90 degrees.

One may observe that the implementation shown in FIG. 15 is similar to that shown in FIG. 14, except that the outer surfaces of the platen 301 have different configurations and the light source system components 304A and 302B are shown to be positioned at different angles. The outer surface 408a of the platen 301 shown in FIG. 14 that is configured to receive a target object resides substantially in a single plane, whereas FIG. 15 shows a recessed area 1505 of the platen 301, including the outer surface 408a and outer surfaces A and B, that are configured to receive a target object. The platen configuration shown in FIG. 15 allows the light 403a and 403b to be provided along the same plane, in opposite directions.

Implementations such as that shown in FIGS. 14 and 15 have the potential advantage that additional light energy may be provided to a target object, as compared to implementations that include only a single light source system. Another potential advantage of implementations like that shown in FIGS. 14 and 15 is that additional light paths into a target object may be provided. The example shown in FIG. 15 provides a further potential advantage, as compared to the example shown in FIG. 14, that relatively less reflected light that is produced by the light 403a and 403b is likely to reach the receiver system 302.

FIG. 16 shows example components of an apparatus according to some additional implementations. As with other figures provided herein, the numbers, types and arrangements of elements shown in FIG. 16 are merely presented by way of example. In this example, the apparatus 300 is an instance of the apparatus 300 shown in FIG. 3. According to this example, the apparatus 300 includes a platen 301, a receiver system 302 and a light source system 304.

In this example, the platen 301 includes a recessed area 1605, including the outer surfaces 408a1 and 408a2, that are configured to receive a target object, such as the finger 115, etc. According to this example, the outer surface 408a1 is perpendicular to, or substantially perpendicular to, the outer surface 408a2. However, in some alternative examples, the outer surface 408a1 may be oriented at an angle that is greater than or less than 90 degrees relative to the outer surface 408a2. In this example, the outer surface 408a2 is at an angle Θ4 to the outer surface 408a3.

According to this example, the light source system 304 is coupled to a surface 408b of the platen 301, which in this instance is parallel to, or substantially parallel to, the outer surface 408a1. In some alternative implementations, the light source system 304 may be proximate, but not coupled to, the surface 408b. In some alternative implementations, the surface 408b may not be parallel to, or substantially parallel to, the outer surface 408a1. In this example, the light source system 304 is configured to provide light 403 to a target object on the outer surface 408a1, if any, via the platen portion 301a. According to this example, at least some of the light 403 travels substantially parallel to the axis 405a, which is perpendicular to, or substantially perpendicular to, the surface 408b and the outer surface 408a1, and is at an angle Θ5 to the outer surface 408a3.

According to this example, the receiver system 302 resides on a surface 408c of the platen 301 that is parallel to, or substantially parallel to, the receiver plane 410. In this example, photoacoustic waves PA are produced in the blood vessel 407 by the light 403. According to this example, at least a portion of the photoacoustic waves PA travel in the platen portion 301b parallel to, or substantially parallel to, the axis 405c, which is parallel to, or substantially parallel to, the axis 405b and the outer surface 408a3 in this instance.

Implementations such as that shown in FIG. 16 have the potential advantage that relatively less reflected light is likely to reach the receiver system 302, in part because the target object may shield the receiver system 302 from at least some reflected light.

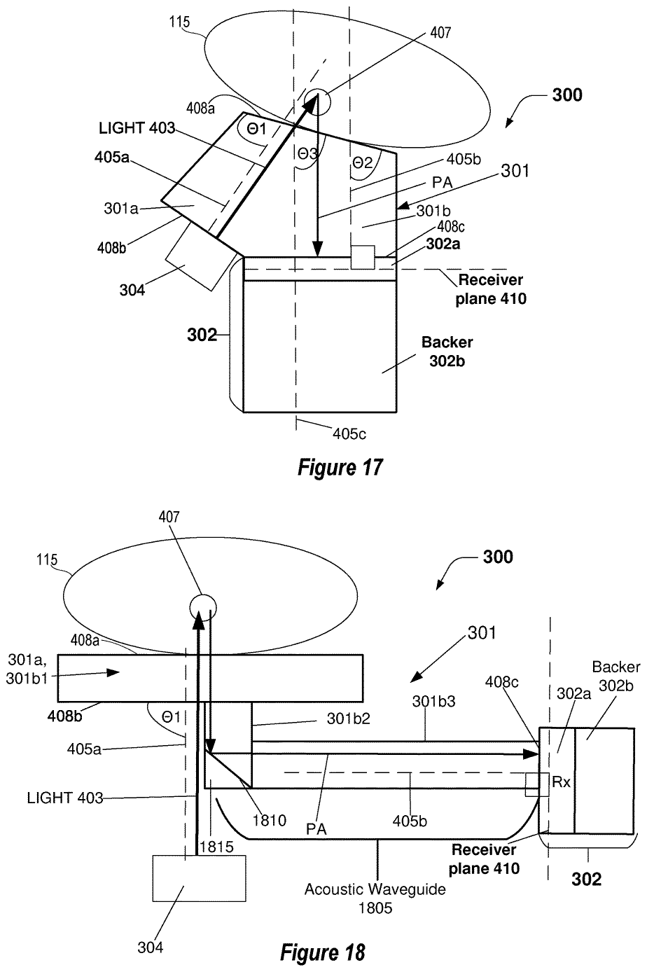

FIG. 17 shows example components of an apparatus according to some additional implementations. As with other figures provided herein, the numbers, types and arrangements of elements shown in FIG. 17 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115, a wrist, etc.

One may observe that in the implementation shown in FIG. 17, the light source system 304 resides on a surface 408b of the platen 301 that is not parallel to, or substantially parallel to, the outer surface 408a. In some alternative implementations, the light source system 304 may be proximate, but not coupled to, the surface 408b. In this example, the light source system 304 is configured to provide light 403 to a target object on the outer surface 408a, if any, via the platen portion 301a. According to this example, at least some of the light 403 travels substantially parallel to the axis 405a, which is perpendicular to, or substantially perpendicular to, the surface 408b and is at an angle Θ1 to the outer surface 408a.

Moreover, in the implementation shown in FIG. 17, the receiver system 302 resides on a surface 408c of the platen 301 that is not parallel to, or substantially parallel to, the outer surface 408a. In this example, the receiver system 302 includes a receiver portion 302a and a backer portion 302b. Here, the receiver plane 410 is parallel to, or substantially parallel to, the surface 408c. The axis 405b, which is normal to the receiver plane 410, forms an angle Θ2 with the outer surface 408a.

In this example, photoacoustic waves PA are produced in the blood vessel 407 by the light 403. According to this example, at least a portion of the photoacoustic waves PA travel in the platen portion 301b parallel to, or substantially parallel to, the axis 405c, which is parallel to, or substantially parallel to, the axis 405b. In this example, the axis 405c forms an angle Θ3 with the outer surface 408a.

In FIG. 17, the outer surface 408a is shown to reside in a single plane. In some alternative implementations, the outer surfaces of the platen 301 that are configured to receive a target object may reside in more than one plane, for example as shown in FIG. 15 or FIG. 16. According to some such implementations, a portion of the outer surface 408a may reside in a plane that is parallel to, or substantially parallel to, the surface 408c of FIG. 17. Alternatively, or additionally, a portion of the outer surface 408a may reside in a plane that is parallel to, or substantially parallel to, the surface 408b of FIG. 17.

FIG. 18 shows example components of an apparatus according to some additional implementations. As with other figures provided herein, the numbers, types and arrangements of elements shown in FIG. 18 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115, a wrist, etc.

One may observe that in the implementation shown in FIG. 18, the light source system 304 is configured to emit at least some of the light 403 through the surface 408b to the outer surface 408a (and to a target object on the surface 408a, if any) along an axis 405a. In some alternative implementations, the light source system 304 may be coupled to the surface 408b. In this example, the light source system 304 is configured to provide light 403 to a target object on the outer surface 408a, if any, via the platen portion 301a, 301b. According to this example, the axis 405a is perpendicular to, or substantially perpendicular to, the outer surface 408a.

In this example, photoacoustic waves PA are produced in the blood vessel 407 by the light 403. According to this example, at least a portion of the photoacoustic waves PA travel in the platen portion 301a,301b parallel to, or substantially parallel to, the axis 405a. According to this example, the apparatus 300 includes an acoustic waveguide 1805. Here, the acoustic waveguide 1805 configured to direct the photoacoustic waves PA, which include ultrasonic waves in this example, towards the receiver system 302, which is, or includes, an ultrasonic receiver system in this instance.

According to this example, the acoustic waveguide 1805 includes platen portions 301b2 and 301b3. In this example, the platen portion 301b3 includes an interface 1810 that is configured to reflect acoustic waves, such as the photoacoustic waves PA. According to this example, the platen portion 301b3 includes an interface 1810 that has a high acoustic impedance contrast. In some such examples, the high acoustic impedance contrast may be caused by air 1815 within the area 1815. If, for example, the platen portions 301b2 and 301b3 are otherwise made of a solid material, such as acrylic, the air/solid material interface 1810 would have a high acoustic impedance contrast.

In the implementation shown in FIG. 18, the receiver system 302 resides on a surface 408c of the platen 301 that is not parallel to, or substantially parallel to, the outer surface 408a. Here, the receiver plane 410 is parallel to, or substantially parallel to, the surface 408c. In this example the axis 405b, which is normal to the receiver plane 410, is parallel to, or substantially parallel to, the outer surface 408a. In other implementations, the axis 405b may not be parallel to, or substantially parallel to, the outer surface 408a.

In this example, the receiver system 302 is, or includes, an ultrasonic receiver system. According to this example, the receiver system 302 includes a receiver portion 302a and a backer portion 302b. As noted elsewhere herein, and as shown in FIG. 17, a backer portion 302b can be advantageous with regard to SNR enhancement, but can substantially add to the total thickness of the receiver system 302. Accordingly, positioning the receiver plane 410 of a relatively thick receiver system 302, which includes a backer portion 302b, such that the receiver plane 410 is normal to, or substantially normal to, the outer surface 408a can advantageously reduce the overall thickness of the apparatus 300 (for example, the thickness along the axis 405a). Put another way, positioning the axis 405b of a receiver system 302 that includes a backer portion 302b such that the axis 405b is parallel to, or substantially parallel to, the outer surface 408a can advantageously reduce the overall thickness of the apparatus 300. According to this example, the acoustic waveguide 1805 allows the receiver system 302 to be positioned in this orientation, or in a similar orientation, by re-directing at least some of the photoacoustic waves PA along the axis 405b.

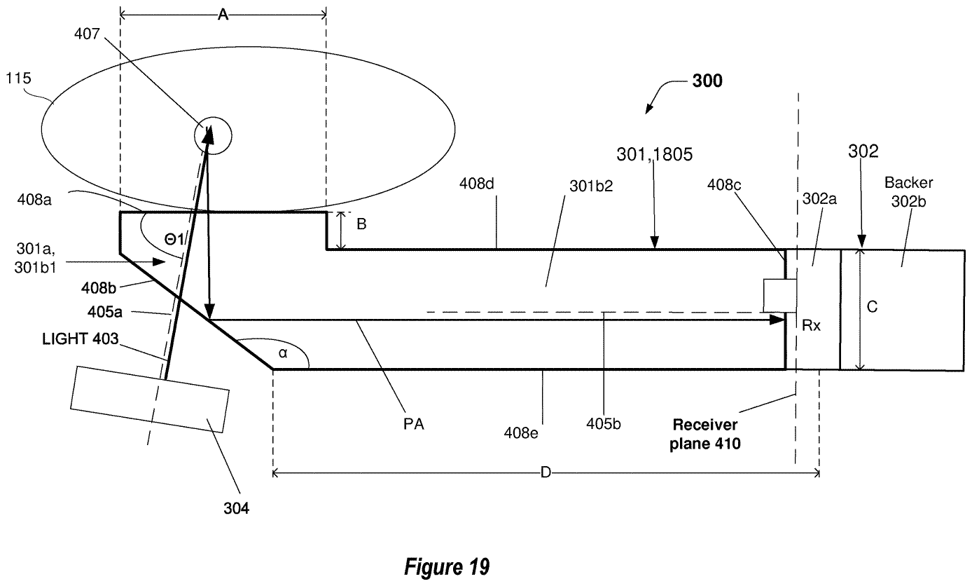

FIG. 19 shows example components of an apparatus according to some additional implementations. As with other figures provided herein, the numbers, types and arrangements of elements shown in FIG. 19 are merely presented by way of example. In this example, the apparatus 300 is an instance of the apparatus 300 shown in FIG. 3. According to this example, the apparatus 300 includes a platen 301, a receiver system 302 and a light source system 304. In this example, an outer surface 408a of the platen 301 is configured to receive a target object, such as the finger 115, a wrist, etc.

In the implementation shown in FIG. 19, the light source system 304 is configured to emit at least some of the light 403 through the surface 408b to the outer surface 408a (and to a target object on the surface 408a, if any) along an axis 405a. According to some implementations, the light source system 304 may be positioned at a desired orientation on a light source system surface, such as the light source system surfaces 304d shown in FIGS. 8 and 9. In some alternative implementations, the light source system 304 may be coupled to the platen 301, for example to the surface 408b. In this example, the light source system 304 is configured to provide light 403 to a target object on the outer surface 408a, if any, via the platen portion 301a, 301b1. According to this example, the axis 405a is at an angle Θ1 relative to the outer surface 408a.

In this example, photoacoustic waves PA are produced in the blood vessel 407 by the light 403. According to this example, at least a portion of the photoacoustic waves PA travel in the platen portion 301b2 parallel to, or substantially parallel to, the axis 405a. According to this example, the platen 301 includes an acoustic waveguide 1805. Here, the acoustic waveguide 1805 configured to direct the photoacoustic waves PA, which include ultrasonic waves in this example, towards the receiver system 302.

According to this example, the acoustic waveguide 1805 includes platen portions 301b2 and 301b3. In this example, the surface 408b includes an interface that is configured to reflect the photoacoustic waves PA. According to this example, the surface 408b corresponds with an interface that has a high acoustic impedance contrast. In this example, the high acoustic impedance contrast is caused by air outside the surface 408b. In this example, the platen portion 301a, 301b1 is made of a solid material, such as acrylic. Therefore, as in the example shown in FIG. 18, the air/solid material interface has a high acoustic impedance contrast.

In the implementation shown in FIG. 19, the receiver system 302 resides on a surface 408c of the platen 301 that is not parallel to, or substantially parallel to, the outer surface 408a. Here, the receiver plane 410 is parallel to, or substantially parallel to, the surface 408c. In this example the axis 405b, which is normal to the receiver plane 410, is parallel to, or substantially parallel to, the outer surface 408a. In other implementations, the axis 405b may not be parallel to, or substantially parallel to, the outer surface 408a.

In this example, the receiver system 302 is, or includes, an ultrasonic receiver system. According to this example, the receiver system 302 includes a receiver portion 302a and a backer portion 302b. As noted elsewhere herein, a backer portion 302b can be advantageous with regard to SNR enhancement, but can substantially add to the total thickness of the receiver system 302. Accordingly, positioning the receiver system 302 as shown in FIG. 19 can advantageously reduce the overall thickness of the apparatus 300. According to this example, the configuration of the platen 301 forms the acoustic waveguide 1805 and allows the receiver system 302 to be positioned in this orientation, or in a similar orientation.

In one non-limiting example, the dimension A may be 4 mm, the dimension B may be 1 mm, the dimension C may be 3 mm, the dimension D may be 10 mm and the angle α may be 135 degrees. Other implementations of the platen 301 may have other configurations, other dimensions, etc.

FIG. 20 shows example components of an apparatus portion according to some additional implementations. As with other figures provided herein, the numbers, types and arrangements of elements shown in FIG. 20 are merely presented by way of example. In this example, portions of an apparatus 300, which is an instance of the apparatus 300 shown in FIG. 3, are shown. According to this example, the portions of the apparatus 300 shown in FIG. 20 include a platen 301 and a receiver system 302.

According to this example, the platen 301 is connected to the receiver system 302 via a conductive double-sided tape (DST) 2001 and conductive ink 2003. In some examples, the conductive DST 2001 may include a copper layer. The conductive ink 2003 may, for example, include silver particles, carbon particles, or combinations thereof, linked to each other by solving agents.

In this example, the receiver system 302 includes an anisotropic conductive film (ACF) 2005, a receiver portion 302a and receiver system circuitry 302e. ACF is an adhesive surface-mount interconnection that allows only the electrical conduction in the thickness direction. In the example shown in FIG. 20, the receiver portion 302a includes a composite piezoelectric material, such as a 1-3 composite, a 2-2 composite, a 3-3 composite, etc. According to this example, the receiver system circuitry 302e includes a flexible printed circuit 2009 and conductive connectors 2007 configured for making an electrical connection between the receiver portion 302a and the flexible printed circuit 2009.

In one non-limiting example, the conductive ink layer may have a thickness of 140 microns and the ACF 2005 may have a thickness of 7 microns. Other implementations of the apparatus 300 may have other configurations, other dimensions, etc.

FIG. 21 shows example components of an apparatus portion according to some implementations. As with other figures provided herein, the numbers, types and arrangements of elements shown in FIG. 21 are merely presented by way of example. In this example, FIG. 21 shows a receiver portion 302a of a receiver system 302.

According to this example, the receiver portion 302a includes a signal electrode 2105, a first layer of piezoelectric copolymer 2115a on a first side of the signal electrode 2105, a second layer of piezoelectric copolymer 2115b on a second and opposite side of the signal electrode 2105, and a grounding structure 2110 that surrounds the signal electrode 2105, the first layer of piezoelectric copolymer 2115a and the second layer of piezoelectric copolymer 2115b. In this example, the first layer of piezoelectric copolymer 2115a and the second layer of piezoelectric copolymer 2115b are each in contact with a portion of the grounding structure 2110. In some examples, the signal electrode 2105, the grounding structure 2110, or both, may include copper or another suitable conductive material.

FIG. 22 shows example components of an apparatus portion according to some alternative implementations. As with other figures provided herein, the numbers, types and arrangements of elements shown in FIG. 22 are merely presented by way of example. In this example, FIG. 22 shows a receiver portion 302a of a receiver system 302.

This example is similar that that shown in FIG. 21. In the example shown in FIG. 22, like that of FIG. 21, the receiver portion 302a includes a signal electrode 2105, a first layer of piezoelectric copolymer 2115a on a first side of the signal electrode 2105, a second layer of piezoelectric copolymer 2115b on a second and opposite side of the signal electrode 2105, and a grounding structure 2110 that surrounds the signal electrode 2105, the first layer of piezoelectric copolymer 2115a and the second layer of piezoelectric copolymer 2115b.

However, in the example shown in FIG. 22, the layers of piezoelectric copolymer 2115a and 2115b do not extend from the signal electrode 2105 to the grounding structure 2110. Instead, in this example, an ACF layer and a conductive layer reside between each layer of piezoelectric copolymer and each grounding layer or signal electrode: the ACF layer 2205a and the conductive layer 2210a reside between the layer of piezoelectric copolymer 2115a and the grounding structure portion 2110a, the ACF layer 2205b and the conductive layer 2210b reside between the layer of piezoelectric copolymer 2115a and the signal electrode 2105, the ACF layer 2205c and the conductive layer 2210c reside between the signal electrode 2105 and the layer of piezoelectric copolymer 2115b, and the ACF layer 2205d and the conductive layer 2210d reside between the layer of piezoelectric copolymer 2115b and the grounding structure portion 2110b. Another minor difference is that in the example shown in FIG. 22, the grounding structure 2110 includes the grounding structure portion 2110a and the grounding structure portion 2110b, which are joined by a conductive adhesive (such as a silver epoxy) in this example.

In one non-limiting example, the grounding structure portion 2110a, the signal electrode 2105 and the grounding structure portion 2110b each may include copper and each may be between 8 microns and 12 microns in thickness, the ACF layers 2205a-2205d each may be between 5 microns and 9 microns in thickness, and the conductive layers 2210a-2210d each may include copper and the layers of piezoelectric copolymer 2115a and 2115b each may be between 300 microns and 400 microns in thickness. In some alternative examples, layers of piezoelectric composite material may replace the layers of piezoelectric copolymer 2115a and 2115b that are shown in FIG. 22.

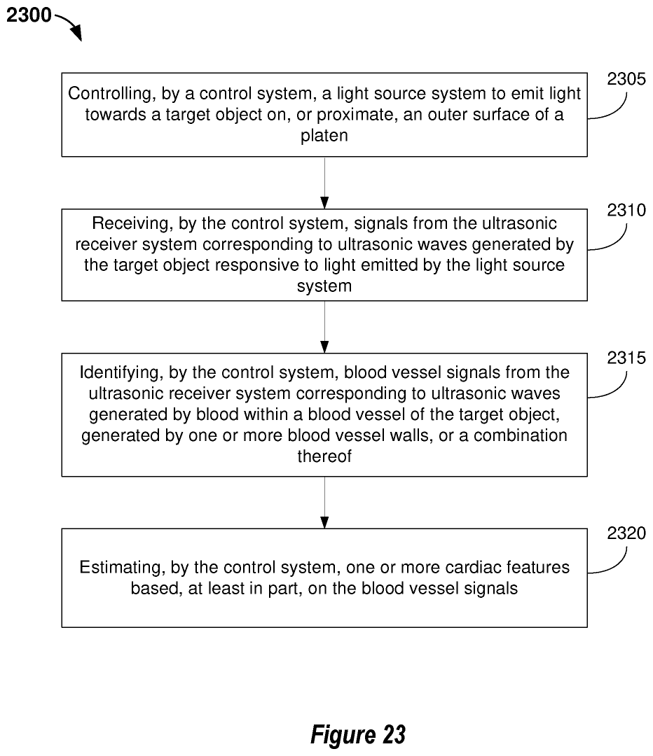

FIG. 23 is a flow diagram that shows examples of some disclosed operations. The blocks of FIG. 23 (and those of other flow diagrams provided herein) may, for example, be performed by the apparatus 300 of FIG. 3 or by a similar apparatus. As with other methods disclosed herein, the method outlined in FIG. 23 may include more or fewer blocks than indicated. Moreover, the blocks of methods disclosed herein are not necessarily performed in the order indicated. In some instances, one or more of the blocks shown in FIG. 23 may be performed concurrently.

In this example, block 2305 involves controlling, by a control system, a light source system to emit light towards a target object on, or proximate, an outer surface of a platen. The target object may be a finger, a wrist, etc., depending on the particular example. According to this example, block 2310 involves receiving, by the control system, signals from the ultrasonic receiver system corresponding to ultrasonic waves generated by the target object responsive to the light emitted by the light source system.

According to this example, block 2315 involves identifying, by the control system, blood vessel signals from the ultrasonic receiver system corresponding to ultrasonic waves generated by blood within a blood vessel of the target object, by one or more blood vessel walls, or combinations thereof. According to some examples, block 2315 may involves identifying, by the control system, arterial signals from the ultrasonic receiver system corresponding to ultrasonic waves generated by blood within an artery of the target object by one or more arterial walls, or combinations thereof. The blood vessel signals may, for example, be identified by implementing a range gate delay (RGD) that corresponds with the expected depth to a blood vessel. Alternatively, or additionally, the arterial signals may be identified according to one or more characteristics of the photoacoustic responses of the blood vessel walls, blood, or a combination thereof.

In this example, block 2320 involves estimating, by the control system, one or more cardiac features based, at least in part, on the blood vessel signals. In some examples, block 2320 may involve estimating a blood pressure based, at least in part, on the blood vessel signals. In some such examples, block 2320 may involve estimating a blood pressure based, at least in part, on arterial signals. According to some examples, block 2320, or another aspect of method 2300, may involve extracting and evaluating heart rate waveform (HRW) features.

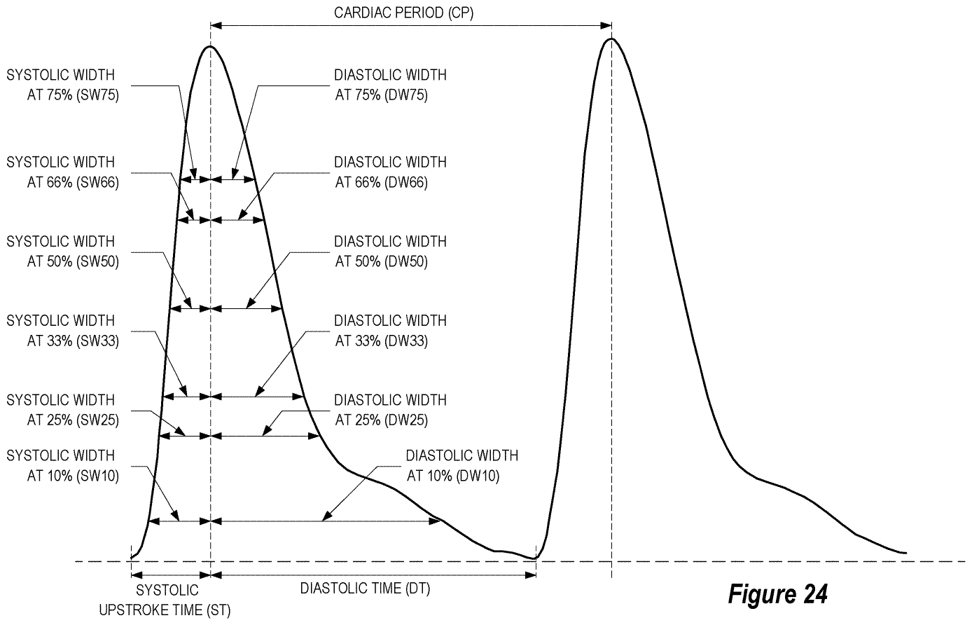

FIG. 24 shows examples of heart rate waveform (HRW) features that may be extracted according to some implementations of the method of FIG. 23. The horizontal axis of FIG. 24 represents time and the vertical axis represents signal amplitude. The cardiac period is indicated by the time between adjacent peaks of the HRW. The systolic and diastolic time intervals are indicated below the horizontal axis. During the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

The HRW features that are illustrated in FIG. 24 pertain to the width of the systolic and/or diastolic portions of the HRW curve at various “heights,” which are indicated by a percentage of the maximum amplitude. For example, the SW50 feature is the width of the systolic portion of the HRW curve at a “height” of 50% of the maximum amplitude. In some implementations, the HRW features used for blood pressure estimation may include some or all of the SW10, SW25, SW33, SW50, SW66, SW75, DW10, DW25, DW33, DW50, DW66 and DW75 HRW features. In other implementations, additional HRW features may be used for blood pressure estimation. Such additional HRW features may, in some instances, include the sum and ratio of the SW and DW at one or more “heights,” e.g., (DW75+SW75), DW75/SW75, (DW66+SW66), DW66/SW66, (DW50+SW50), DW50/SW50, (DW33+SW33), DW33/SW33, (DW25+SW25), DW25/SW25 and/or (DW10+SW10), DW10/SW10. Other implementations may use yet other HRW features for blood pressure estimation. Such additional HRW features may, in some instances, include sums, differences, ratios and/or other operations based on more than one “height,” such as (DW75+SW75)/(DW50+SW50), (DW50+SW50/(DW10+SW10), etc.

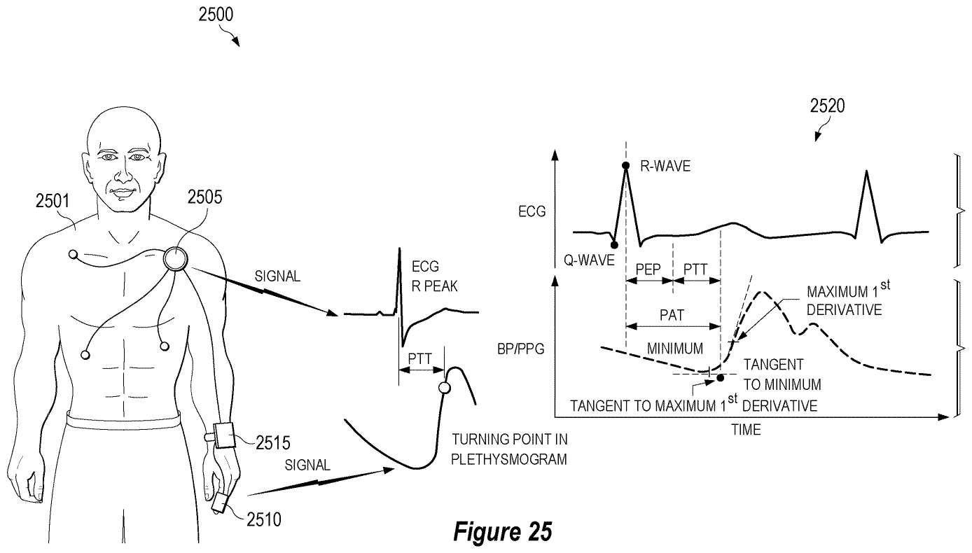

FIG. 25 shows examples of devices that may be used in a system for estimating blood pressure based, at least in part, on pulse transit time (PTT). As with other figures provided herein, the numbers, types and arrangements of elements are merely presented by way of example. According to this example, the system 2500 includes at least two sensors. In this example, the system 2500 includes at least an electrocardiogram sensor 2505 and a device 2510 that is configured to be mounted on a finger of the person 2501. In this example, the device 2510 is, or includes, an apparatus configured to perform at least some PAPG methods disclosed herein. For example, the device 2510 may be, or may include, the apparatus 300 of FIG. 3 or a similar apparatus.

As noted in the graph 2520, the PAT includes two components, the pre-ejection period (PEP, the time needed to convert the electrical signal into a mechanical pumping force and isovolumetric contraction to open the aortic valves) and the PTT. The starting time for the PAT can be estimated based on the QRS complex—an electrical signal characteristic of the electrical stimulation of the heart ventricles. As shown by the graph 2520, in this example the beginning of a pulse arrival time (PAT) may be calculated according to an R-Wave peak measured by the electrocardiogram sensor 2505 and the end of the PAT may be detected via analysis of signals provided by the device 2510. In this example, the end of the PAT is assumed to correspond with an intersection between a tangent to a local minimum value detected by the device 2510 and a tangent to a maximum slope/first derivative of the sensor signals after the time of the minimum value.

There are many known algorithms for blood pressure estimation based on the PTT and/or the PAT, some of which are summarized in Table 1 and described in the corresponding text on pages 5-10 of Sharma, M., et al., Cuff-Less and Continuous Blood Pressure Monitoring: a Methodological Review (“Sharma”), in Multidisciplinary Digital Publishing Institute (MDPI) Technologies 2017, 5, 21, both of which are hereby incorporated by reference.

Some previously-disclosed methods have involved calculating blood pressure according to one or more of the equations shown in Table 1 of Sharma, or other known equations, based on a PTT and/or PAT measured by a sensor system that includes a PPG sensor. As noted above, some disclosed PAPG-based implementations are configured to distinguish artery HRWs from other HRWs. Such implementations may provide more accurate measurements of the PTT and/or PAT, relative to those measured by a PPG sensor. Therefore, disclosed PAPG-based implementations may provide more accurate blood pressure estimations, even when the blood pressure estimations are based on previously-known formulae.

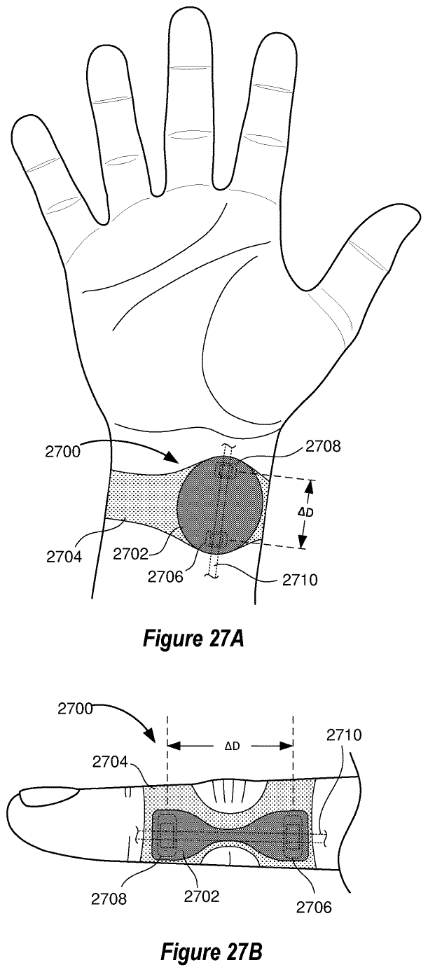

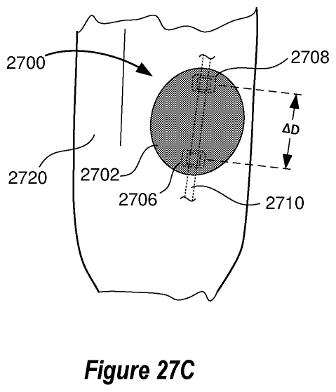

Other implementations of the system 2500 may not include the electrocardiogram sensor 2505. In some such implementations, the device 2515, which is configured to be mounted on a wrist of the person 2501, may be, or may include, an apparatus configured to perform at least some PAPG methods disclosed herein. For example, the device 2515 may be, or may include, the apparatus 200 of FIG. 2 or a similar apparatus. According to some such examples, the device 2515 may include a light source system and two or more ultrasonic receivers. One example is described below with reference to FIG. 27A. In some examples, the device 2515 may include an array of ultrasonic receivers.

In some implementations of the system 2500 that do not include the electrocardiogram sensor 2505, the device 2510 may include a light source system and two or more ultrasonic receivers. One example is described below with reference to FIG. 27B.

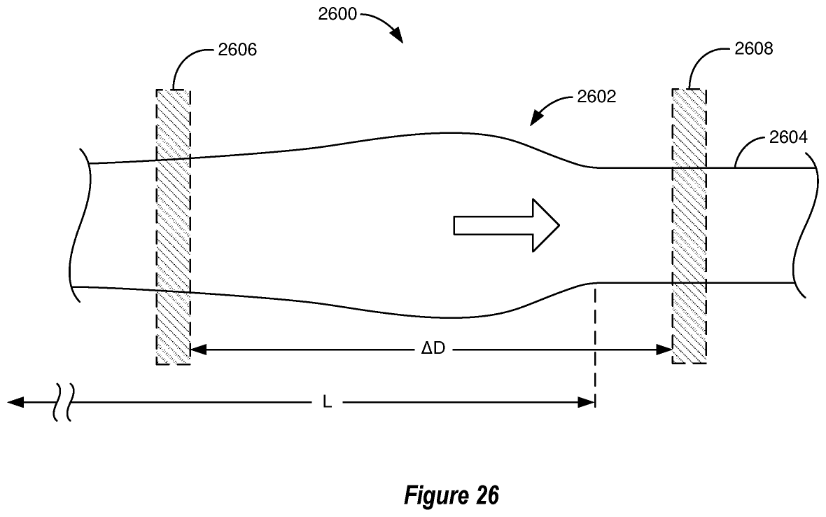

FIG. 26 shows a cross-sectional side view of a diagrammatic representation of a portion of an artery 2600 through which a pulse 2602 is propagating. The block arrow in FIG. 26 shows the direction of blood flow and pulse propagation. As diagrammatically shown, the propagating pulse 2602 causes strain in the arterial walls 2604, which is manifested in the form of an enlargement in the diameter (and consequently the cross-sectional area) of the arterial walls—referred to as “distension.” The spatial length L of an actual propagating pulse along an artery (along the direction of blood flow) is typically comparable to the length of a limb, such as the distance from a subject's shoulder to the subject's wrist or finger, and is generally less than one meter (m). However, the length L of a propagating pulse can vary considerably from subject to subject, and for a given subject, can vary significantly over durations of time depending on various factors. The spatial length L of a pulse will generally decrease with increasing distance from the heart until the pulse reaches capillaries.

As described above, some particular implementations relate to devices, systems and methods for estimating blood pressure or other cardiovascular characteristics based on estimates of an arterial distension waveform. The terms “estimating,” “measuring,” “calculating,” “inferring,” “deducing,” “evaluating,” “determining” and “monitoring” may be used interchangeably herein where appropriate unless otherwise indicated. Similarly, derivations from the roots of these terms also are used interchangeably where appropriate; for example, the terms “estimate,” “measurement,” “calculation,” “inference” and “determination” also are used interchangeably herein. In some implementations, the pulse wave velocity (PWV) of a propagating pulse may be estimated by measuring the pulse transit time (PTT) of the pulse as it propagates from a first physical location along an artery to another more distal second physical location along the artery. It will be appreciated that this PTT is different from the PTT that is described above with reference to FIG. 15. However, either version of the PTT may be used for the purpose of blood pressure estimation. Assuming that the physical distance ΔD between the first and the second physical locations is ascertainable, the PWV can be estimated as the quotient of the physical spatial distance ΔD traveled by the pulse divided by the time (PTT) the pulse takes in traversing the physical spatial distance ΔD. Generally, a first sensor positioned at the first physical location is used to determine a starting time (also referred to herein as a “first temporal location”) at which point the pulse arrives at or propagates through the first physical location. A second sensor at the second physical location is used to determine an ending time (also referred to herein as a “second temporal location”) at which point the pulse arrives at or propagates through the second physical location and continues through the remainder of the arterial branch. In such examples, the PTT represents the temporal distance (or time difference) between the first and the second temporal locations (the starting and the ending times).

The fact that measurements of the arterial distension waveform are performed at two different physical locations implies that the estimated PWV inevitably represents an average over the entire path distance ΔD through which the pulse propagates between the first physical location and the second physical location. More specifically, the PWV generally depends on a number of factors including the density of the blood ρ, the stiffness E of the arterial wall (or inversely the elasticity), the arterial diameter, the thickness of the arterial wall, and the blood pressure. Because both the arterial wall elasticity and baseline resting diameter (for example, the diameter at the end of the ventricular diastole period) vary significantly throughout the arterial system, PWV estimates obtained from PTT measurements are inherently average values (averaged over the entire path length ΔD between the two locations where the measurements are performed).

In traditional methods for obtaining PWV, the starting time of the pulse has been obtained at the heart using an electrocardiogram (ECG) sensor, which detects electrical signals from the heart. For example, the starting time can be estimated based on the QRS complex—an electrical signal characteristic of the electrical stimulation of the heart ventricles. In such approaches, the ending time of the pulse is typically obtained using a different sensor positioned at a second location (for example, a finger). As a person having ordinary skill in the art will appreciate, there are numerous arterial discontinuities, branches, and variations along the entire path length from the heart to the finger. The PWV can change by as much as or more than an order of magnitude along various stretches of the entire path length from the heart to the finger. As such, PWV estimates based on such long path lengths are unreliable.

In various implementations described herein, PTT estimates are obtained based on measurements (also referred to as “arterial distension data” or more generally as “sensor data”) associated with an arterial distension signal obtained by each of a first arterial distension sensor 2606 and a second arterial distension sensor 2608 proximate first and second physical locations, respectively, along an artery of interest. In some particular implementations, the first arterial distension sensor 2606 and the second arterial distension sensor 2608 are advantageously positioned proximate first and second physical locations between which arterial properties of the artery of interest, such as wall elasticity and diameter, can be considered or assumed to be relatively constant. In this way, the PWV calculated based on the PTT estimate is more representative of the actual PWV along the particular segment of the artery. In turn, the blood pressure P estimated based on the PWV is more representative of the true blood pressure. In some implementations, the magnitude of the distance ΔD of separation between the first arterial distension sensor 2606 and the second arterial distension sensor 2608 (and consequently the distance between the first and the second locations along the artery) can be in the range of about 1 centimeter (cm) to tens of centimeters—long enough to distinguish the arrival of the pulse at the first physical location from the arrival of the pulse at the second physical location, but close enough to provide sufficient assurance of arterial consistency. In some specific implementations, the distance ΔD between the first and the second arterial distension sensors 2606 and 2608 can be in the range of about 1 cm to about 30 cm, and in some implementations, less than or equal to about 20 cm, and in some implementations, less than or equal to about 10 cm, and in some specific implementations less than or equal to about 5 cm. In some other implementations, the distance ΔD between the first and the second arterial distension sensors 2606 and 2608 can be less than or equal to 1 cm, for example, about 0.1 cm, about 0.25 cm, about 0.5 cm or about 0.75 cm. By way of reference, a typical PWV can be about 15 meters per second (m/s). Using an ambulatory monitoring device in which the first and the second arterial distension sensors 2606 and 2608 are separated by a distance of about 5 cm, and assuming a PWV of about 15 m/s implies a PTT of approximately 3.3 milliseconds (ms).

The value of the magnitude of the distance ΔD between the first and the second arterial distension sensors 2606 and 2608, respectively, can be preprogrammed into a memory within a monitoring device that incorporates the sensors (for example, such as a memory of, or a memory configured for communication with, the control system 306 that is described above with reference to FIG. 3). As will be appreciated by a person of ordinary skill in the art, the spatial length L of a pulse can be greater than the distance ΔD from the first arterial distension sensor 2606 to the second arterial distension sensor 2608 in such implementations. As such, although the diagrammatic pulse 2602 shown in FIG. 26 is shown as having a spatial length L comparable to the distance between the first arterial distension sensor 2606 and the second arterial distension sensor 2608, in actuality each pulse can typically have a spatial length L that is greater and even much greater than (for example, about an order of magnitude or more than) the distance ΔD between the first and the second arterial distension sensors 2606 and 2608.

### Sensing Architecture and Topology

In some implementations of the ambulatory monitoring devices disclosed herein, both the first arterial distension sensor 2606 and the second arterial distension sensor 2608 are sensors of the same sensor type. In some such implementations, the first arterial distension sensor 2606 and the second arterial distension sensor 2608 are identical sensors. In such implementations, each of the first arterial distension sensor 2606 and the second arterial distension sensor 2608 utilizes the same sensor technology with the same sensitivity to the arterial distension signal caused by the propagating pulses, and has the same time delays and sampling characteristics. In some implementations, each of the first arterial distension sensor 2606 and the second arterial distension sensor 2608 is configured for photoacoustic plethysmography (PAPG) sensing, e.g., as disclosed elsewhere herein. Some such implementations include a light source system and two or more ultrasonic receivers, which may be instances of the light source system 304 and the receiver system 302 of FIG. 3. In some implementations, each of the first arterial distension sensor 2606 and the second arterial distension sensor 2608 is configured for ultrasound sensing via the transmission of ultrasonic signals and the receipt of corresponding reflections. In some alternative implementations, each of the first arterial distension sensor 2606 and the second arterial distension sensor 2608 may be configured for impedance plethysmography (IPG) sensing, also referred to in biomedical contexts as bioimpedance sensing. In various implementations, whatever types of sensors are utilized, each of the first and the second arterial distension sensors 2606 and 2608 broadly functions to capture and provide arterial distension data indicative of an arterial distension signal resulting from the propagation of pulses through a portion of the artery proximate to which the respective sensor is positioned. For example, the arterial distension data can be provided from the sensor to a processor in the form of voltage signal generated or received by the sensor based on an ultrasonic signal or an impedance signal sensed by the respective sensor.

As described above, during the systolic phase of the cardiac cycle, as a pulse propagates through a particular location along an artery, the arterial walls expand according to the pulse waveform and the elastic properties of the arterial walls. Along with the expansion is a corresponding increase in the volume of blood at the particular location or region, and with the increase in volume of blood an associated change in one or more characteristics in the region. Conversely, during the diastolic phase of the cardiac cycle, the blood pressure in the arteries decreases and the arterial walls contract. Along with the contraction is a corresponding decrease in the volume of blood at the particular location, and with the decrease in volume of blood an associated change in the one or more characteristics in the region.

In the context of bioimpedance sensing (or impedance plethysmography), the blood in the arteries has a greater electrical conductivity than that of the surrounding or adjacent skin, muscle, fat, tendons, ligaments, bone, lymph or other tissues. The susceptance (and thus the permittivity) of blood also is different from the susceptances (and permittivities) of the other types of surrounding or nearby tissues. As a pulse propagates through a particular location, the corresponding increase in the volume of blood results in an increase in the electrical conductivity at the particular location (and more generally an increase in the admittance, or equivalently a decrease in the impedance). Conversely, during the diastolic phase of the cardiac cycle, the corresponding decrease in the volume of blood results in an increase in the electrical resistivity at the particular location (and more generally an increase in the impedance, or equivalently a decrease in the admittance).

A bioimpedance sensor generally functions by applying an electrical excitation signal at an excitation carrier frequency to a region of interest via two or more input electrodes, and detecting an output signal (or output signals) via two or more output electrodes. In some more specific implementations, the electrical excitation signal is an electrical current signal injected into the region of interest via the input electrodes. In some such implementations, the output signal is a voltage signal representative of an electrical voltage response of the tissues in the region of interest to the applied excitation signal. The detected voltage response signal is influenced by the different, and in some instances time-varying, electrical properties of the various tissues through which the injected excitation current signal is passed. In some implementations in which the bioimpedance sensor is operable to monitor blood pressure, heartrate or other cardiovascular characteristics, the detected voltage response signal is amplitude-and phase-modulated by the time-varying impedance (or inversely the admittance) of the underlying arteries, which fluctuates synchronously with the user's heartbeat as described above. To determine various biological characteristics, information in the detected voltage response signal is generally demodulated from the excitation carrier frequency component using various analog or digital signal processing circuits, which can include both passive and active components.

In some examples incorporating ultrasound sensors, measurements of arterial distension may involve directing ultrasonic waves into a limb towards an artery, for example, via one or more ultrasound transducers. Such ultrasound sensors also are configured to receive reflected waves that are based, at least in part, on the directed waves. The reflected waves may include scattered waves, specularly reflected waves, or both scattered waves and specularly reflected waves. The reflected waves provide information about the arterial walls, and thus the arterial distension.

In some implementations, regardless of the type of sensors utilized for the first arterial distension sensor 2606 and the second arterial distension sensor 2608, both the first arterial distension sensor 2606 and the second arterial distension sensor 2608 can be arranged, assembled or otherwise included within a single housing of a single ambulatory monitoring device. As described above, the housing and other components of the monitoring device can be configured such that when the monitoring device is affixed or otherwise physically coupled to a subject, both the first arterial distension sensor 2606 and the second arterial distension sensor 2608 are in contact with or in close proximity to the skin of the user at first and second locations, respectively, separated by a distance ΔD, and in some implementations, along a stretch of the artery between which various arterial properties can be assumed to be relatively constant. In various implementations, the housing of the ambulatory monitoring device is a wearable housing or is incorporated into or integrated with a wearable housing. In some specific implementations, the wearable housing includes (or is connected with) a physical coupling mechanism for removable non-invasive attachment to the user. The housing can be formed using any of a variety of suitable manufacturing processes, including injection molding and vacuum forming, among others. In addition, the housing can be made from any of a variety of suitable materials, including, but not limited to, plastic, metal, glass, rubber and ceramic, or combinations of these or other materials. In particular implementations, the housing and coupling mechanism enable full ambulatory use. In other words, some implementations of the wearable monitoring devices described herein are noninvasive, not physically-inhibiting and generally do not restrict the free uninhibited motion of a subject's arms or legs, enabling continuous or periodic monitoring of cardiovascular characteristics such as blood pressure even as the subject is mobile or otherwise engaged in a physical activity. As such, the ambulatory monitoring device facilitates and enables long-term wearing and monitoring (for example, over days, weeks or a month or more without interruption) of one or more biological characteristics of interest to obtain a better picture of such characteristics over extended durations of time, and generally, a better picture of the user's health.

In some implementations, the ambulatory monitoring device can be positioned around a wrist of a user with a strap or band, similar to a watch or fitness/activity tracker. FIG. 27A shows an example ambulatory monitoring device 2700 designed to be worn around a wrist according to some implementations. In the illustrated example, the monitoring device 2700 includes a housing 2702 integrally formed with, coupled with or otherwise integrated with a wristband 2704. The first and the second arterial distension sensors 2706 and 2708 may, in some instances, each include an instance of the ultrasonic receiver system 302 and a portion of the light source system 304 that are described above with reference to FIG. 3. In this example, the ambulatory monitoring device 2700 is coupled around the wrist such that the first and the second arterial distension sensors 2706 and 2708 within the housing 2702 are each positioned along a segment of the radial artery 2710 (note that the sensors are generally hidden from view from the external or outer surface of the housing facing the subject while the monitoring device is coupled with the subject, but exposed on an inner surface of the housing to enable the sensors to obtain measurements through the subject's skin from the underlying artery). Also as shown, the first and the second arterial distension sensors 2706 and 2708 are separated by a fixed distance ΔD. In some other implementations, the ambulatory monitoring device 2700 can similarly be designed or adapted for positioning around a forearm, an upper arm, an ankle, a lower leg, an upper leg, or a finger (all of which are hereinafter referred to as “limbs”) using a strap or band.

FIG. 27B shows an example ambulatory monitoring device 2700 designed to be worn on a finger according to some implementations. The first and the second arterial distension sensors 2706 and 2708 may, in some instances, each include an instance of the ultrasonic receiver 302 and a portion of the light source system 304 that are described above with reference to FIG. 3.

In some other implementations, the ambulatory monitoring devices disclosed herein can be positioned on a region of interest of the user without the use of a strap or band. For example, the first and the second arterial distension sensors 2706 and 2708 and other components of the monitoring device can be enclosed in a housing that is secured to the skin of a region of interest of the user using an adhesive or other suitable attachment mechanism (an example of a “patch” monitoring device).

FIG. 27C shows an example ambulatory monitoring device 2700 designed to reside on an earbud according to some implementations. According to this example, the ambulatory monitoring device 2700 is coupled to the housing of an earbud 2720. The first and second arterial distension sensors 2706 and 2708 may, in some instances, each include an instance of the ultrasonic receiver 302 and a portion of the light source system 304 that are described above with reference to FIG. 3.

Implementation examples are described in the following numbered clauses:
1. An apparatus, including: a platen; a light source system configured to provide light to a target object on an outer surface of the platen, where the light source system is configured to direct the light along a first axis oriented at a first angle relative to the outer surface of the platen; and an ultrasonic receiver system configured to receive ultrasonic waves generated by the target object responsive to the light from the light source system, the ultrasonic receiver system including one or more receiver elements residing in a receiver plane, a normal to the receiver plane being oriented along a second axis at a second angle relative to the outer surface of the platen.
2. The apparatus of clause 1, where the second angle is approximately 90 degrees.
3. The apparatus of clause 1, where the second angle is in a range from 20 degrees to 50 degrees.
4. The apparatus of any one of clauses 1-3, where at least a portion of the platen is configured to direct the ultrasonic waves generated by the target object along a third axis that is at a third angle relative to the outer surface of the platen.
5. The apparatus of clause 4, where the third axis is parallel, or substantially parallel, to the second axis.
6. The apparatus of clause 4, where the third angle is different from the first angle.
7. The apparatus of any one of clauses 1-6, where the light source system includes one or more light-directing elements configured to direct light from the light source system towards the target object along the first axis.
8. The apparatus of clause 7, where the one or more light-directing elements includes a diffraction grating.
9. The apparatus of clause 7 or clause 8, where the one or more light-directing elements includes a lens.
10. The apparatus of any one of clauses 1-9, where the light source system includes a light source system surface having a normal that is parallel, or substantially parallel, to the first axis, where a light source of the light source system resides on the light source system surface.
11. The apparatus of any one of clauses 1-10, where a first platen portion residing between the light source system and the outer surface of the platen may have a first platen portion thickness that is less than a second platen portion thickness of a second platen portion residing between at least one receiver element of the ultrasonic receiver system and the outer surface of the platen.
12. The apparatus of any one of clauses 1-11, where a first platen portion residing between the light source system and the outer surface of the platen may have a first platen portion thickness that is greater than a second platen portion thickness of a second platen portion residing between at least one receiver element of the ultrasonic receiver system and the outer surface of the platen.
13. The apparatus of any one of clauses 1-12, further including an acoustic waveguide configured to direct ultrasonic waves toward at least one receiver element of the ultrasonic receiver system.
14. The apparatus of any one of clauses 1-13, where a first distance traversed through the platen by the light from the light source system is less than a second distance traversed by the ultrasonic waves through the platen and to at least one receiver element of the ultrasonic receiver system.
15. The apparatus of any one of clauses 1-14, where a platen portion configured to receive the light from the light source system is also configured to reflect the ultrasonic waves generated by the target object towards at least one receiver element of the ultrasonic receiver system.
16. The apparatus of clause 15, where a first distance traversed by light from the light source system through the platen portion to the target object is less than a second distance traversed by the ultrasonic waves from the platen portion to at least one receiver element of the ultrasonic receiver system.
17. The apparatus of any one of clauses 1-16, where the platen includes a recessed area configured to receive a digit.
18. The apparatus of clause 1, where the first axis is perpendicular to, or substantially perpendicular to, the outer surface.
19. The apparatus of clause 18, where at least a portion of the platen is configured to direct the ultrasonic waves generated by the target object along a third axis that is at a third angle relative to the outer surface of the platen, where the second axis is parallel, or substantially parallel, to the third axis and where the first angle is different from the third angle.
20. The apparatus of clause 19, where a first portion of the platen is configured to direct a first portion of the ultrasonic waves generated by the target object along the third axis and a second portion of the platen is configured to direct a second portion of the ultrasonic waves generated by the target object along a fourth axis at a fourth angle relative to the outer surface, the third angle being different from the first angle and the fourth angle.
21. The apparatus of clause 20, where the ultrasonic receiver system includes: a first receiver element residing in a first receiver plane that is substantially perpendicular to the third axis; and a second receiver element residing in a second receiver plane that is substantially perpendicular to the fourth axis.
22. The apparatus of any one of clauses 1-21, where at least a portion of the platen is configured to direct the ultrasonic waves generated by the target object along a third axis that is at a third angle relative to the outer surface of the platen and where the third axis is perpendicular to, or substantially perpendicular to, the outer surface.
23. The apparatus of clause 22, where the second axis is parallel, or substantially parallel, to the third axis and where the first angle is different from the third angle.
24. The apparatus of clause 23, where the light source system includes: a first light source portion configured to direct first light towards the target object along the first axis; and a second light source portion configured to direct second light towards the target object along a fourth axis at a fourth angle relative to the outer surface of the platen, the third angle being different from the first angle and the fourth angle.
25. The apparatus of any one of clauses 1-24, further including acoustic isolation material residing between the light source system and at least a portion of the ultrasonic receiver system.
26. The apparatus of any one of clauses 1-25, further including electromagnetic noise suppression material proximate at least a portion of the ultrasonic receiver system, proximate conductive material attached to at least a portion of the ultrasonic receiver system, or a combination thereof.
27. The apparatus of any one of clauses 1-26, where the apparatus is configured for attachment to a human wrist.
28. The apparatus of clause 27, where the light source system configured to provide light to one or more arteries within the human wrist.
29. The apparatus of any one of clauses 1-26, where the light source system is configured to provide light to one or more arteries within a human finger.
30. The apparatus of any one of clauses 1-29, where the platen, the light source system, or a combination thereof, is configured for transmitting light in a range from 400 to 1000 nanometers.
31. The apparatus of any one of clauses 1-30, where the light source system is configured to provide pulses of light having pulse widths in a range from 50 to 500 nanoseconds.
32. The apparatus of any one of clauses 1-31, where at least a portion of the ultrasonic receiver system includes a composite piezoelectric material.
33. The apparatus of any one of clauses 1-32, where at least a portion of the ultrasonic receiver system includes a conductive layer, a first piezoelectric layer proximate a first side of the conductive layer and a second piezoelectric layer proximate a second side of the conductive layer.
34. The apparatus of clause 33, where the first piezoelectric layer and the second piezoelectric layer include a piezoelectric copolymer, a piezoelectric composite, or a combination thereof.
35. The apparatus of clause 33, further including a first electrical grounding layer portion and a second electrical grounding layer portion, where the first piezoelectric layer resides between the first electrical grounding layer portion and the conductive layer and the second piezoelectric layer resides between the second electrical grounding layer portion and the conductive layer.
36. The apparatus of any one of clauses 1-35, where at least a portion of the platen has an acoustic impedance that is configured to approximate an acoustic impedance of a substance proximate the portion of platen.
37. The apparatus of any one of clauses 1-36, further including a control system configured to: control the light source system to emit light; receive signals from the ultrasonic receiver corresponding to the ultrasonic waves generated by the target object; identify blood vessel signals from the ultrasonic receiver corresponding to ultrasonic waves generated by blood within a blood vessel of the target object, generated by one or more blood vessel walls, or a combination thereof; and estimate one or more cardiac features based, at least in part, on the blood vessel signals.
38. The apparatus of any one of clauses 1-37, further including one or more mirror layers configured to reflect light away from one or more portions of the ultrasonic receiver system.
39. The apparatus of any one of clauses 1-38, where an outer surface of the platen, or a layer residing on an outer surface of the platen, has an acoustic impedance that is configured to approximate the acoustic impedance of human skin.
40. The apparatus of any one of clauses 1-39, further including a layer residing between the platen and the one or more receiver elements, an acoustic impedance of the layer being in an acoustic impedance range between an acoustic impedance of the platen and an acoustic impedance of the one or more receiver elements.
41. The apparatus of any one of clauses 1-40, further including one or more anti-reflective layers.
42. The apparatus of clause 41, where at least one of the one or more anti-reflective layers resides proximate the outer surface of the platen.
43. The apparatus of any one of clauses 1-42, where at least a portion of the platen includes an acoustic lens system.
44. The apparatus of clause 43, where the acoustic lens system includes a spherical lens, a cylindrical lens, or both.
45. The apparatus of clause 43 or clause 44, where the acoustic lens system resides on, or proximate, an outer surface of the platen.
46. The apparatus of any one of clauses 1-45, further comprising one or more optical waveguides.
47. The apparatus of clause 46, where at least a portion of one of the one or more optical waveguides resides in a portion of the platen.

As used herein, a phrase referring to “at least one of” a list of items refers to any combination of those items, including single members. As an example, “at least one of: a, b, or c” is intended to cover: a, b, c, a-b, a-c, b-c, and a-b-c.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general purpose single-or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, e.g., a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also may be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium, such as a non-transitory medium. The processes of a method or algorithm disclosed herein may be implemented in a processor-executable software module which may reside on a computer-readable medium. Computer-readable media include both computer storage media and communication media including any medium that may be enabled to transfer a computer program from one place to another. Storage media may be any available media that may be accessed by a computer. By way of example, and not limitation, non-transitory media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those having ordinary skill in the art, and the generic principles defined herein may be applied to other implementations without departing from the spirit or scope of this disclosure. Thus, the disclosure is not intended to be limited to the implementations shown herein, but is to be accorded the widest scope consistent with the claims, the principles and the novel features disclosed herein. The word “exemplary” is used exclusively herein, if at all, to mean “serving as an example, instance, or illustration.” Any implementation described herein as “exemplary” is not necessarily to be construed as preferred or advantageous over other implementations.

Certain features that are described in this specification in the context of separate implementations also may be implemented in combination in a single implementation. Conversely, various features that are described in the context of a single implementation also may be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, other implementations are within the scope of the following claims. In some cases, the actions recited in the claims may be performed in a different order and still achieve desirable results.

It will be understood that unless features in any of the particular described implementations are expressly identified as incompatible with one another or the surrounding context implies that they are mutually exclusive and not readily combinable in a complementary and/or supportive sense, the totality of this disclosure contemplates and envisions that specific features of those complementary implementations may be selectively combined to provide one or more comprehensive, but slightly different, technical solutions. It will therefore be further appreciated that the above description has been given by way of example only and that modifications in detail may be made within the scope of this disclosure.

Various modifications to the implementations described in this disclosure may be readily apparent to those skilled in the art, and the generic principles defined herein may be applied to other implementations without departing from the spirit or scope of this disclosure. Thus, the following claims are not intended to be limited to the implementations shown herein, but are to be accorded the widest scope consistent with this disclosure, the principles and the novel features disclosed herein.

Additionally, certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, various ones of the described and illustrated operations can itself include and collectively refer to a number of sub-operations. For example, each of the operations described above can itself involve the execution of a process or algorithm. Furthermore, various ones of the described and illustrated operations can be combined or performed in parallel in some implementations. Similarly, the separation of various system components in the implementations described above should not be understood as requiring such separation in all implementations. As such, other implementations are within the scope of the following claims. In some cases, the actions recited in the claims can be performed in a different order and still achieve desirable results.

What is claimed is:

1. An apparatus, comprising:
a platen;
a light source system configured to provide light to a target object on an outer surface of the platen, wherein the light source system is configured to direct the light along a first axis oriented at a first angle relative to the outer surface of the platen;
an ultrasonic receiver system configured to receive ultrasonic waves generated by the target object responsive to the light from the light source system, the ultrasonic receiver system including one or more receiver elements residing in a receiver plane, a normal to the receiver plane being oriented along a second axis at a second angle relative to the outer surface of the platen; and
an acoustic waveguide configured to direct the ultrasonic waves toward at least one receiver element of the ultrasonic receiver system.

2. The apparatus of claim 1, wherein the second angle is approximately 90 degrees.

3. The apparatus of claim 1, wherein at least a portion of the platen is configured to direct the ultrasonic waves generated by the target object along a third axis that is at a third angle relative to the outer surface of the platen.

4. The apparatus of claim 3, wherein the third axis is parallel, or substantially parallel, to the second axis.

5. The apparatus of claim 1, wherein the light source system includes one or more light-directing elements configured to direct light from the light source system towards the target object along the first axis.

6. The apparatus of claim 5, wherein the one or more light-directing elements includes a lens.

7. The apparatus of claim 1, wherein a first platen portion residing between the light source system and the outer surface of the platen has a first platen portion thickness that is greater than a second platen portion thickness of a second platen portion residing between at least one receiver element of the ultrasonic receiver system and the outer surface of the platen.

8. The apparatus of claim 1, wherein the platen includes the acoustic waveguide.

9. The apparatus of claim 1, wherein a first distance traversed through the platen by the light from the light source system to the target object is less than a second distance traversed by the ultrasonic waves from the target object through the platen and to at least one receiver element of the ultrasonic receiver system.

10. The apparatus of claim 1, further comprising acoustic isolation material residing between the light source system and at least a portion of the ultrasonic receiver system.

11. The apparatus of claim 1, wherein the light source system is configured to provide light to one or more arteries within a human wrist.

12. The apparatus of claim 1, wherein the light source system is configured to provide light to one or more arteries within a human finger.

13. The apparatus of claim 1, wherein the platen, the light source system, or a combination thereof, is configured for transmitting light in a range from 400 to 1000 nanometers.

14. The apparatus of claim 1, wherein the light source system is configured to provide pulses of light having pulse widths in a range from 50 to 500 nanoseconds.

15. The apparatus of claim 1, wherein at least a portion of the ultrasonic receiver system includes a composite piezoelectric material.

16. The apparatus of claim 1, further comprising a control system configured to:
control the light source system to emit light;
receive ultrasonic receiver signals from the ultrasonic receiver system corresponding to the ultrasonic waves generated by the target object;
identify blood vessel signals from the ultrasonic receiver signals corresponding to ultrasonic waves generated by blood within a blood vessel of the target object, generated by one or more blood vessel walls, or a combination thereof; and
estimate one or more cardiac features based, at least in part, on the blood vessel signals.

17. The apparatus of claim 1, further comprising one or more mirror layers configured to reflect light away from one or more portions of the ultrasonic receiver system.

18. The apparatus of claim 1, wherein an outer surface of the platen, or a layer residing on an outer surface of the platen, has an acoustic impedance that is configured to approximate the acoustic impedance of human skin.

19. The apparatus of claim 1, further comprising a layer residing between the platen and the one or more receiver elements, an acoustic impedance of the layer being in an acoustic impedance range between an acoustic impedance of the platen and an acoustic impedance of the one or more receiver elements.

20. The apparatus of claim 1, wherein at least a portion of the platen includes an acoustic lens system.
